(19) United States
(12) Patent Application Publication
(10) Pub. No.: US 2014/0254758 A1
(43) Pub. Date: Sep. 11, 2014

(54) X-RAY IMAGING SYSTEM

(71) Applicant: CANON KABUSHIKI KAISHA, Tokyo (JP)

(72) Inventor: Akio Saigusa, Tama-shi (JP)

(73) Assignee: CANON KABUSHIKI KAISHA, Tokyo (JP)

(21) Appl. No.: 14/198,183

(22) Filed: Mar. 5, 2014

(30) Foreign Application Priority Data
Mar. 6, 2013 (JP) 2013-044402

(51) Int. Cl. A61B 6/00

(52) U.S. Cl. CPC A61B 6/545; A61B 6/542; A61B 6/461
USPC 378/62

(57) ABSTRACT

A management apparatus for managing radiation imaging receives function information about a radiation imaging apparatus and communication information about communication between the radiation imaging apparatus and a radiation generation apparatus. Based on the function information or communication information received, an operation setting unit in the management apparatus sets an operation mode of the radiation imaging apparatus; and a control unit perform control corresponding to radiation imaging based on the set operation mode.

### BACKGROUND OF THE INVENTION

1. Field of the Invention

The present invention relates to a wireless X-ray imaging apparatus and an X-ray imaging system which perform analog-to-digital (A/D) conversion on a captured X-ray image for digitization and transmit data of the digitized X-ray image by using a wireless communication unit.

2. Description of the Related Art

X-ray imaging systems that digitize an X-ray image captured by an X-ray imaging apparatus and apply image processing to the digitized X-ray image to generate a sharper X-ray image have heretofore been commercialized.

X-ray imaging is typically performed with an X-ray imaging apparatus fixed and installed on a pedestal or a flat-bed pedestal. To perform X-ray imaging with a higher degree of freedom, an X-ray imaging apparatus may be used in a free position without being mechanically fixed.

To meet such needs, X-ray imaging apparatuses of wireless type have been commercialized. The X-ray imaging apparatuses are made wireless to improve the degree of freedom of installation.

Such X-ray imaging apparatuses need synchronization control between X-ray irradiation timing of an X-ray generation apparatus and a charge accumulation period of the X-ray imaging apparatuses. As a result, connectable X-ray generation apparatuses have sometimes been limited.

Digital X-ray imaging apparatuses having an X-ray automatic detection (automatic trigger) function have recently been commercialized. Such digital X-ray imaging apparatuses can be easily incorporated into even an X-ray imaging system including an X-ray generation apparatus that has no function for synchronization control on the X-ray irradiation timing (for example, an X-ray imaging system using a computed radiography (CR) device as an X-ray imaging apparatus).

A wireless X-ray imaging system connects an X-ray imaging apparatus and a control personal computer (PC) by wireless communication, whereby digital X-ray image data and control commands for the X-ray imaging apparatus are transmitted and received. For example, if a wireless local area network (LAN) is used as a wireless communication unit, a client (X-ray imaging apparatus) attempting to connect to an access point needs to establish connection with the access point by using wireless LAN connection related information (for example, a communication method such as the Institute of Electrical and Electronics Engineers (IEEE) 802.11n, a physical channel, an extended service set identifier (ESSID), and an encryption key).

A method is known in which the control PC transmits the wireless LAN connection related information about the connected access point to the X-ray imaging apparatus by using an entry device (for example, a short-range wireless communication unit) different from the wireless LAN. The X-ray imaging apparatus establishes wireless LAN connection by using the received wireless LAN connection related information.

With the advancement of the X-ray imaging apparatuses, it has become possible to use one X-ray imaging apparatus in combination with a plurality of X-ray rooms and portable X-ray generation apparatuses (mobile X-ray apparatuses).

Some radiation imaging systems include a radiation generation apparatus having a communication function, and some do not. Radiation imaging apparatuses may be equipped with different functions. Appropriate settings need to be made according to such differences in the system configuration, or there occurs a problem that radiation irradiation cannot be performed or that radiation irradiation can be performed but the radiation imaging apparatus cannot obtain a proper image.

### SUMMARY OF THE INVENTION

According to an aspect of the present invention, a management apparatus of radiation imaging by a radiation imaging apparatus and a radiation generation apparatus includes an obtaining unit configured to obtain function information about the radiation imaging apparatus and communication information about communication between the radiation imaging apparatus and the radiation generation apparatus, an operation setting unit configured to set an operation mode of the radiation imaging apparatus based on the function information or communication information obtained by the obtaining unit, and a control unit configured to perform control corresponding to radiation imaging based on the set operation mode.

Further features of the present invention will become apparent from the following description of exemplary embodiments with reference to the attached drawings.

### BRIEF DESCRIPTION OF THE DRAWINGS

FIG. 1 is a schematic diagram illustrating an apparatus configuration example of an X-ray imaging system according to a first exemplary embodiment of the present invention.

FIG. 2 is a block diagram illustrating a configuration of the X-ray imaging system according to the first exemplary embodiment of the present invention.

FIG. 3 is a flowchart illustrating an operation when registering an X-ray imaging apparatus with the X-ray imaging system according to the first exemplary embodiment of the present invention.

FIG. 4 is a schematic diagram illustrating an example of a general configuration of an X-ray imaging system according to a second exemplary embodiment of the present invention.

FIG. 5 is a block diagram illustrating a configuration of the apparatuses in the X-ray imaging system according to the second exemplary embodiment of the present invention.

FIG. 6 is a flowchart illustrating an operation when registering an X-ray imaging apparatus with the X-ray imaging system according to the second exemplary embodiment of the present invention.

FIG. 7 is a schematic diagram illustrating communication setting processing between radiation imaging apparatuses and an imaging control apparatus.

FIG. 8 is a block diagram of a radiation imaging system according to another exemplary embodiment.

FIG. 9 is a block diagram of a radiation sensor and its peripheral circuits.

FIG. 10 is a diagram illustrating an example of table information used for operation mode setting.

FIG. 11, which is composed of FIGS. 11A and 11B, is a flowchart illustrating a flow of processing for setting an operation mode of the radiation imaging system.

FIG. 12, which is composed of FIGS. 12A and 12B, is a flowchart illustrating a flow of radiation imaging processing in a set operation mode.

FIG. 13 is a block diagram illustrating a hardware configuration example of a radiation imaging system.

### DESCRIPTION OF THE EMBODIMENTS

Various exemplary embodiments, features, and aspects of the invention will be described in detail below with reference to the drawings. A radiation imaging system according to an exemplary embodiment uses an X-ray generation apparatus 402 that includes an X-ray irradiation switch 311 and emits X-rays in response to pressing of the X-ray irradiation switch 311. Alternatively, the radiation imaging system may use the X-ray generation apparatus 402 that further includes an X-ray control apparatus communication circuit (synchronization control unit) 208 and emits X-rays in synchronization with an X-ray imaging apparatus 101 in response to the pressing of the X-ray irradiation switch 311.

The radiation imaging system according to the exemplary embodiment further includes the X-ray imaging apparatus 101, a registration communication device (entry device) 317, and a control PC 301. The X-ray imaging apparatus 101 includes a central processing unit (CPU) 110 which performs control for synchronization with an X-ray irradiation timing of the X-ray generation apparatus 402. The registration communication device 317 performs communication for registering the X-ray imaging apparatus 101 with the radiation imaging system. The control PC 301 is connected with the registration communication device 317. The control PC 301 performs control for registering the X-ray imaging apparatus 101 with the radiation imaging system and imaging control of the registered X-ray imaging apparatus 101.

The X-ray imaging apparatus 101 or the control PC 301 functions as a management apparatus of the radiation imaging system. If the X-ray imaging apparatus 101 functions as the management apparatus, an operation mode control circuit 113 of the X-ray imaging apparatus 101 obtains communication information about communication between the X-ray imaging apparatus 101 and the X-ray generation apparatus 402. The operation mode control circuit 113 sets operation modes of the X-ray imaging apparatus 101 and the control PC 301 based on the obtained communication information in combination with function information about the X-ray imaging apparatus 101. In addition, a drive control circuit 105 of the X-ray imaging apparatus 101 performs imaging control corresponding to an operation mode similarly set by a CPU 206.

If the control PC 301 functions as the management apparatus, an operation mode setting unit 318 of the control PC 301 sets the operation mode of the X-ray imaging apparatus 101 and makes an imaging control setting of the control PC 301 via the registration communication device 317 based on the function information about the X-ray imaging apparatus 101 and the communication information about communication between the X-ray imaging apparatus 101 and the X-ray generation apparatus 402. In addition, an imaging control unit 303 of the control PC 301 performs control for causing the X-ray imaging apparatus 101 to perform radiation imaging based on the set operation mode.

Among possible operation modes is an X-ray automatic detection (auto trigger) mode. In the X-ray automatic detection mode, a photoelectric conversion element (radiation sensor) 102 shifts to an accumulation state in response to detection of radiation irradiation by a detection circuit which detects a start of radiation irradiation. The accumulation state refers to a state in which the photoelectric conversion element 102 can convert radiations into an electrical signal. In the X-ray automatic detection mode, the X-ray imaging apparatus 101 detects the X-ray irradiation and shifts to a charge accumulation period, whereby X-ray irradiation timing control between the X-ray imaging apparatus 101 and the X-ray generation apparatus 402 becomes unnecessary. In the X-ray automatic detection mode, the X-ray imaging apparatus 101 shifts to the charge accumulation period according to the X-ray irradiation timing. The X-ray generation apparatus 402 can thus emit X-rays in arbitrary timing.

Another operation mode is an X-ray manual synchronization (timer imaging) mode in which the photoelectric conversion element 102 of the X-ray imaging apparatus 101 shifts to the charge accumulation state in response to a lapse of a predetermined time from a user's instruction input. In the X-ray manual synchronization mode, for example, an operator determines the charge accumulation period of the X-ray imaging apparatus 101, and presses the X-ray irradiation switch 311 to control the X-ray irradiation timing. In the X-ray manual synchronization mode, a display unit such as a display 310 is used to notify the operator that the X-ray imaging apparatus 101 has shifted to the charge accumulation period. The operator presses the X-ray irradiation switch 311 at the notified timing, thereby matching the charge accumulation period with the X-ray irradiation timing.

Another operation mode is an X-ray synchronization mode in which the X-ray imaging apparatus 101 establishes synchronization with the X-ray generation apparatus 402 via communication with the foregoing X-ray control apparatus communication circuit 208, thereby matching operation timing between the X-ray imaging apparatus 101 and the X-ray generation apparatus 402. In the X-ray synchronization mode, the photoelectric conversion element 102 shifts to the charge accumulation state in an interlocking manner with the pressing of the X-ray irradiation switch 311. The X-ray imaging apparatus 101 communicates with the X-ray generation apparatus 402 to emit X-rays during the charge accumulation period, whereby the X-ray irradiation timing is synchronized. In the X-ray synchronization mode, the X-ray generation apparatus 402 notifies the X-ray imaging apparatus 101 of the pressing of the X-ray irradiation switch 311 and the completion timing of the X-ray irradiation. The X-ray imaging apparatus 101 notifies the X-ray generation apparatus 402 of the timing when the photoelectric conversion element 102 shifts to the charge accumulation period. In such a manner, the charge accumulation period is matched with the X-ray irradiation timing.

Suppose that the correspondence between the function information about the X-ray imaging apparatus 101 and the communication information about the X-ray generation apparatus 402 is inconsistent and an appropriate operation mode cannot be set. In such a case, the CPU 110 or the imaging control unit 303 performs control to prohibit imaging by the X-ray imaging apparatus 101. For example, the CPU 110 or the imaging control unit 303 performs control so that the X-ray imaging apparatus 101 cannot perform communication. Alternatively, a display control unit 307 causes the display 310 to notify the operator of the inconsistency instead of or in addition to the prohibition of the imaging. In such a manner, imaging with an inappropriate system configuration can be prohibited or reduced.

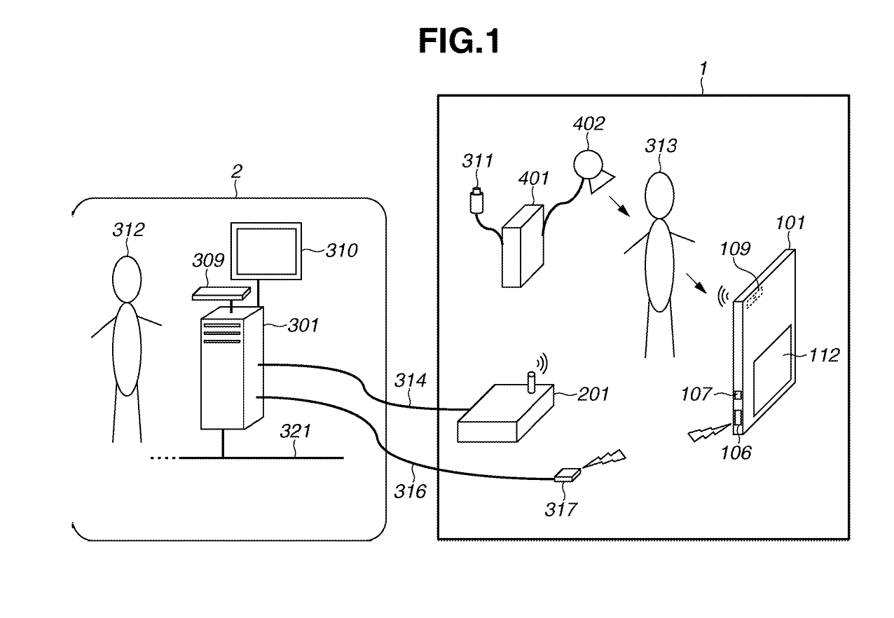

A radiation imaging system according to a first exemplary embodiment will be described with reference to FIG. 1. FIG. 1 is a schematic diagram illustrating an apparatus configuration example of a wireless X-ray imaging system according to the first exemplary embodiment. The first exemplary embodiment deals with an X-ray imaging system that has no function for synchronization control on X-ray irradiation timing.

An X-ray room 1 is intended to perform X-ray imaging using X-ray irradiation. A control room 2 is located near the X-ray room 1.

The X-ray imaging apparatus 101 generates digital X-ray image data in response to X-rays. A power supply control circuit 112 is intended to supply power to the X-ray imaging apparatus 101. A wireless communication circuit 109 is intended to perform wireless LAN communication. A short-range wireless communication circuit 106 has a communication range shorter than that of a wireless LAN. A registration switch 107 is intended to start short-range wireless communication.

An access point 201 is intended to perform wireless communication with the X-ray imaging apparatus 101. A connection cable 314 connects the access point 201 and the control PC 301 in a wired manner. A connection cable 403 connects the control PC 301 and an X-ray control apparatus 401 in a wired manner.

The display 310 is used to display image-processed X-ray image data and a graphical user interface (GUI). The control PC 301 controls the X-ray imaging apparatus 101 and the X-ray generation apparatus 402, and performs image processing. A backbone network 321 connects the control PC 301. An example of the backbone network 321 is an in-house LAN. The entry device 317 is intended to perform short-range wireless communication with the X-ray imaging apparatus 101. A connection cable 316 connects the control PC 301 and the entry device 317 in a wired manner.

An operation of the X-ray imaging system will be described. An operator 312 initially performs an operation for registering the X-ray imaging apparatus 101 with the X-ray imaging system. The operator 312 presses the registration switch 107 of the X-ray imaging apparatus 101 to start short-range wireless communication between the short-range wireless communication circuit 106 of the X-ray imaging apparatus 101 and the entry device 317. The X-ray imaging apparatus 101 receives function information via the short-range wireless communication with the entry device 317 of the control PC 301, whereby the operation mode of the X-ray imaging apparatus 101 and the X-ray generation apparatus 402 is automatically set.

Next, the control PC 301 transmits wireless LAN connection related information (for example, a communication method such as IEEE 802.11n, a physical channel, an ESSID, and an encryption key) to the X-ray imaging apparatus 101 via the short-range wireless communication of the entry device 317. The X-ray imaging apparatus 101 makes settings according to the received wireless LAN connection related information, and establishes wireless LAN communication connection with the access point 201.

Next, the operator 312 inputs patient information and an imaging region of a patient 313 into the control PC 301. The patient information includes an identification data (ID), name, and the date of birth of the patient 313. After the input of the imaging region, the operator 312 fixes the position of the patient 313 and the X-ray imaging apparatus 101.

Completing the imaging preparation, the operator 312 presses the X-ray irradiation switch 311. When the X-ray irradiation switch 311 is pressed, the X-ray generation apparatus 402 irradiates the patient 313 with X-rays. The emitted X-rays are transmitted through the patient 313 and incident on the X-ray imaging apparatus 101.

The X-ray imaging apparatus 101 converts the incident X-rays into visible light, and then detects the visible light as an X-ray image signal by using the photoelectric conversion element 102. The X-ray imaging apparatus 101 drives the photoelectric conversion element 102 to read the X-ray image signal. The X-ray imaging apparatus 101 converts the analog signal into a digital signal by using an A/D conversion circuit 104, whereby digital X-ray image data is obtained. The obtained digital X-ray image data is transferred from the X-ray imaging apparatus 101 to the control PC 301 via the access point 201.

The control PC 301 performs image processing on the received digital X-ray image data. The control PC 301 displays, on the display 310, an X-ray image based on the image-processed X-ray image data.

The operation of the X-ray imaging system from the registration of the X-ray imaging apparatus 101 with the X-ray imaging system by the operator 312 to the display of an X-ray image of the patient 313 on the display 310 has been described above.

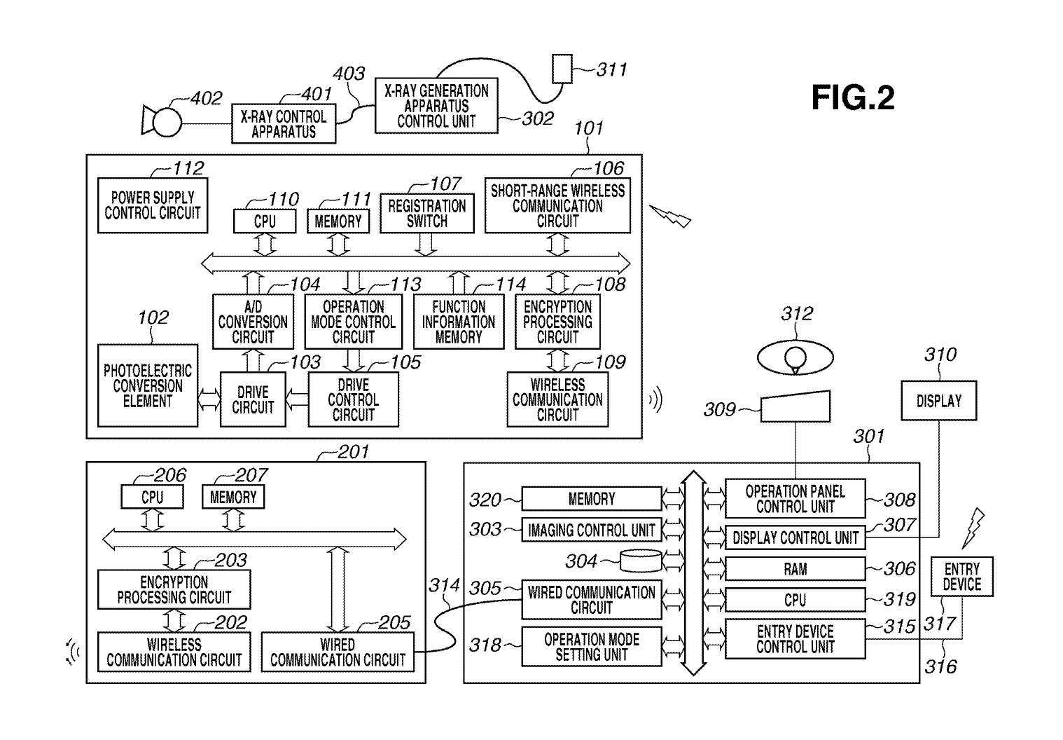

Next, a detailed configuration of each of the apparatuses will be described with reference to the block diagram in FIG. 2.

The X-ray imaging system includes the X-ray imaging apparatus 101, the X-ray control apparatus 401, the X-ray generation apparatus 402, the access point 201, the entry device 317, the control PC 301, an operation panel 309, the display 310, and the X-ray irradiation switch 311.

The X-ray imaging apparatus 101 includes the CPU 110, a memory 111, the photoelectric conversion element 102, a drive circuit 103, the A/D conversion circuit 104, the drive control circuit 105, the short-range wireless communication circuit 106, the registration switch 107, the operation mode control circuit 113, a function information memory 114, an encryption processing circuit 108, the wireless communication circuit 109, and the power supply control circuit 112.

The CPU 110 controls the entire X-ray imaging apparatus 101 by using programs and various types of data stored in the memory 111.

The memory 111 stores, for example, programs and various types of data that the CPU 110 uses when performing processing. The memory 111 further stores various types of data obtained by the processing of the CPU 110 and X-ray image data.

The photoelectric conversion element 102 includes a two-dimensional array of a plurality of pixels (for example, 2688×2688 pixels having 160-μm resolution). The photoelectric conversion element 102 is mainly made of amorphous silicon. The photoelectric conversion element 102 receives X-rays converted into visible light, and detects the visible light as an X-ray image signal.

The drive circuit 103 drives the photoelectric conversion element 102 to perform processing for reading the X-ray image signal.

The drive circuit 103 controls the photoelectric element 102 to shift to an accumulation state for accumulating charge or a state for not accumulating charge. Examples of the state for not accumulating charge include a sleep state where no voltage is applied to the photoelectric conversion element 102, a sensor wait state where voltage is applied to the photoelectric conversion element 102, and a sensor reading state where the photoelectric conversion element 102 is driven to read the X-ray image signal.

The A/D conversion circuit 104 converts the analog X-ray image signal read by the drive circuit 103 into a digital X-ray image signal. The A/D conversion circuit 104 stores the digital X-ray image signal into the memory 111 as X-ray image data.

The drive control circuit 105 controls the drive circuit 103 based on instructions from the operation mode control circuit 113.

The short-range wireless communication circuit 106 communicates with the control PC 301 via the entry device 317. Examples of the short-range wireless communication circuit 106 and the entry devices 317 are infrared communication units. Other examples include communication units that can perform communication based on specifications such as near field communication (NFC), Bluetooth (registered trademark), ZigBee, and Transfer Jet. The short-range wireless communication circuit 106 receives wireless communication parameters. The short-range wireless communication circuit 106 also receives communication information indicating whether communication can be performed with a radiation generation apparatus. In another exemplary embodiment, the short-range wireless communication circuit 106 transmits function information.

The registration switch 107 is a push switch to be operated by the operator 312. If the operator 312 presses the registration switch 107, the registration of the X-ray imaging apparatus 101 with the X-ray imaging system is started.

The operation mode control circuit 113 sets an operation mode based on the function information about the X-ray imaging apparatus 101. In another exemplary embodiment, the operation mode control circuit 113 controls the drive control circuit 105 according to an operation mode specified by a command from the control PC 301.

Information about functions (operation modes) supported by the X-ray imaging apparatus 101 is recorded in the function information memory 114. The memory 111 may be used instead of the function information memory 114. The function information refers to information that indicates whether the X-ray imaging apparatus 101 has functions needed to perform the X-ray automatic detection mode, the X-ray manual synchronization mode, or the X-ray synchronization mode. For example, in the case of the X-ray automatic detection mode, the function information indicates whether the X-ray imaging apparatus 101 includes a detection circuit for detecting a start of radiation irradiation and can detect radiations. The CPU 110 determines the presence or absence of the detection circuit based on model information and unit configuration information about the X-ray imaging apparatus 101, which are stored in the memory 111. For example, if the detection circuit or the photoelectric conversion element 102 cannot detect radiations due to low battery level, the X-ray imaging apparatus 101 is determined not to have the function of performing the X-ray automatic detection mode. The absence of the function is then written to the function information memory 114. For the X-ray manual synchronization mode or the X-ray synchronization mode, if a software program for performing communication with the X-ray generation apparatus 402 or for receiving an instruction from outside is implemented, the X-ray imaging apparatus 101 is determined to have the function. If no such software program is implemented, the X-ray imaging apparatus 101 is determined not to have the function. In such cases, the presence or absence of the functions depends on the configuration of the programs stored in the memory 111. Configuration information about the programs is written to the memory 111 at the time of factory shipment or program update, for example. The CPU 110 may collect the configuration information from the memory 111 upon periodic checks. According to such configuration information, the CPU 110 obtains and writes function information to the function information memory 114. In such a manner, the CPU 110 collects the function information based on the presence or absence of units in the X-ray imaging apparatus 101, the configuration of programs, and the state of components. The information about the units, the configuration of the programs, and the state of the components may be simply stored as function information. Based on such information, the CPU 110 obtains information indicating whether each mode is available and the information may be used as function information instead.

The function information is written to the function information memory 114 at the time of factory shipment. Alternatively, the CPU 110 performs control to monitor the state of each of the components of the X-ray imaging apparatus 101 as appropriate, and according to the state, sequentially updates the values written in the function information memory 114. The function information is obtained from the function information memory 114 by the control of the CPU 110.

The encryption processing circuit 108 encrypts communication data and outputs the encrypted communication data to the wireless communication circuit 109 at the time of transmission. At the time of reception, the encryption processing circuit 108 decrypts encrypted communication data received by the wireless communication circuit 109.

The wireless communication circuit 109 transmits the encrypted communication data input from the encryption processing circuit 108, and outputs the received communication data to the encryption processing circuit 108.

The power supply control circuit 112 includes a battery and a direct-current-to-direct-current (DCDC) converter. The power supply control circuit 112 supplies power to the foregoing circuits.

The access point 201 includes a wireless communication circuit 202, an encryption processing circuit 203, a wired communication circuit 205, the CPU 206, and a memory 207.

The CPU 206 controls the entire access point 201 by using programs and various types of data stored in the memory 207.

The memory 207 stores, for example, programs and various types of data that the CPU 206 uses when performing processing. The memory 207 further stores various types of data obtained by the processing of the CPU 206 and wireless communication data.

The encryption processing circuit 203 encrypts communication data and outputs the encrypted communication data to the wireless communication circuit 202 at the time of transmission. At the time of reception, the encryption processing circuit 203 decrypts encrypted communication data received by the wireless communication circuit 202.

The wireless communication circuit 202 transmits the encrypted communication data input from the encryption processing circuit 203, and outputs the received communication data to the encryption processing circuit 203.

The wired communication circuit 205 controls communication of various types of data and various types of information performed between the access point 201 and the control PC 301.

The control PC 301 includes an X-ray generation apparatus control unit 302, the imaging control unit 303, an external storage device 304, a wired communication circuit 305, a CPU 319, a random access memory (RAM) 306, the display control unit 307, an operation panel control unit 308, an entry device control unit 315, the operation mode setting unit 318, and a memory 320. In the present exemplary embodiment, the control PC 301 can operate in the X-ray synchronization mode, the X-ray automatic detection mode, and the X-ray manual synchronization mode by using programs stored in the memory 320.

The X-ray generation apparatus control unit 302 performs control related to the generation of X-rays by the X-ray generation apparatus 402 based on an imaging instruction from the operator 312. The X-ray generation apparatus control unit 302 has no function for synchronization control on the X-ray irradiation timing. If the X-ray irradiation switch 311 is pressed, the X-ray generation apparatus 402 starts X-ray irradiation regardless of the state of the X-ray imaging apparatus 101.

The imaging control unit 303 performs control related to X-ray imaging on the X-ray imaging apparatus 101 based on an imaging instruction from the operator 312 and an operation mode set by the operation mode setting unit 318.

The external storage device 304 includes, for example, a hard disk. The external storage device 304 stores various programs, various types of data, or various types of information.

The wired communication circuit 305 controls communication of various types of data and various types of information performed between the control PC 301 and the access point 201.

The communication cable 314 connects the access point 201 and the control PC 301 to allow communication therebetween.

The CPU 319 controls the entire control PC 301 by using programs and various types of data stored in the RAM 306.

The RAM 306 temporarily stores various types of data and various types of information necessary for the processing of the control PC 301.

The display control unit 307 performs various types of control related to the display of the display 310.

The operation panel control unit 308 performs various types of control related to the operation panel 309. Examples include switching the display of the operation panel 309 according to an operation of the operation panel 309 by the operator 312.

The operation panel 309 is operated by the operator 312. The operation panel 309 is used for inputting instructions input from the operator 312 to the control PC 301.

The X-ray irradiation switch 311 is operated by the operator 312. When the operator 312 presses the X-ray irradiation switch 311, an imaging instruction is input to the X-ray generation apparatus control unit 302 and the imaging control unit 303, whereby X-ray imaging is started.

The display 310 displays various images and information based on control by the display control unit 307.

The imaging control unit 303, the display control unit 307, the operation panel control unit 308, the entry device control unit 315, and the operation mode setting unit 318 may be implemented by using dedicated hardware circuits such as a field-programmable gate array (FPGA). Alternatively, the CPU 319 may read, from the memory 320, a program for implementing each of the functions of such units, load the program into the RAM 306, and execute a command included in the program to implement the function of each unit.

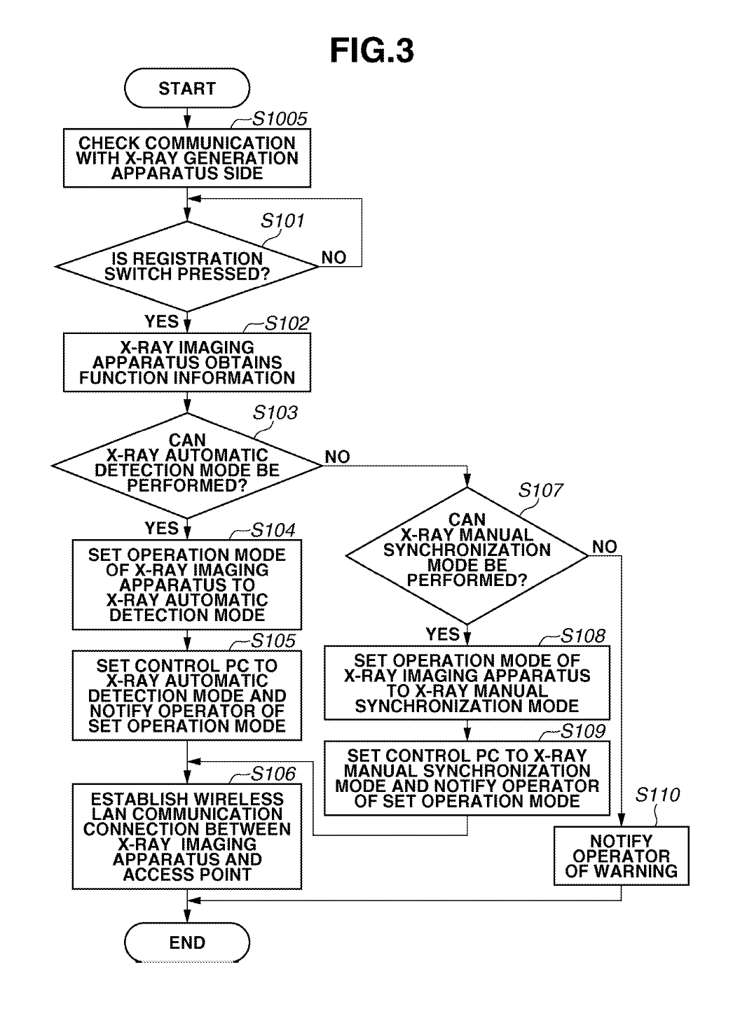

A procedure for setting the operation mode of the X-ray imaging apparatus 101 and the control PC 301 according to the present exemplary embodiment will be described in detail. FIG. 3 is a flowchart illustrating an operation when registering the X-ray imaging apparatus 101 with the X-ray imaging system. To implement the operation of the X-ray imaging apparatus 101, the CPU 110 reads, from the memory 111, a program for implementing the following operations and executes a command similarly loaded and stored in a work memory of the memory 111, thereby controlling the components of the X-ray imaging apparatus 101. The subject of the processing in the flowchart is the CPU 110 unless otherwise specified.

In step S1005, the control PC 301 checks communication with the X-ray generation 402 side. For example, the control PC 301 may check the communication by transmitting a ping command to a specific internet protocol (IP) address and receiving a pong. The control PC 301 may refer to information in the memory 320 and check whether communication has been established with the X-ray generation apparatus 402 side. In a subsequent step, the information indicating whether the communication has been established is transmitted to the X-ray imaging apparatus 101 as communication information via the entry device 317.

In step S101, the CPU 110 detects pressing of the registration switch 107 of the X-ray imaging apparatus 101. If the CPU 110 detects that the registration switch 107 is pressed (YES in step S101), the processing proceeds to step S102. (The CPU 110 waits until the registration switch 107 is pressed.)

In step S102, the X-ray imaging apparatus 101 obtains function information. In the present exemplary embodiment, the X-ray imaging apparatus 101 performs the setting of the operation mode. The CPU 110 therefore obtains the function information recorded in the function image memory 114. In step S102, the CPU 110 further receives the communication information about communication between the X-ray imaging apparatus 101 and the X-ray generation apparatus 402 side from the control PC 301 via the entry device 317.

In step S103, the CPU 110 determines the operation mode based on the function information and the communication information. The present exemplary embodiment assumes the case where the communication with the X-ray generation apparatus 402 side has not been established. The CPU 110 thus determines that an operation in the X-ray synchronization mode is impossible, and subsequently determines whether the X-ray automatic detection mode can be performed. Here, the CPU 110 refers to the function information and determines whether the X-ray imaging apparatus 101 includes the detection circuit. In another example, the CPU 110 may further determine whether there is a corresponding program and whether the components of the X-ray imaging apparatus 101 are in an appropriate state. If the CPU 110 determines that a start of X-ray irradiation can be detected (YES in step S103), the processing proceeds to step S104. If the CPU 110 determines that the start of the X-ray irradiation cannot be detected (NO in step S103), the processing proceeds to step S107.

In step S104, the operation mode control circuit 113 sets the operation mode of the X-ray imaging apparatus 101 to the X-ray automatic detection mode. The operation mode control circuit 113 stores information indicating the setting into the memory 111, and transmits the information to the drive control circuit 105. In the drive control circuit 105, circuitry for executing control corresponding to the X-ray automatic detection mode is activated. The drive control circuit 105 may be configured so that its power supply can be partly turned off. In such a case, during the period in which the operation mode is set to the X-ray automatic detection mode, control circuit portions for implementing the functions corresponding to the X-ray synchronization mode and the X-ray manual synchronization mode can be powered off for power saving even after imaging is started or even if the control circuit corresponding to the X-ray automatic detection mode is powered on. Similar control can also be performed for power saving if the drive control circuit 105 includes a plurality of control circuits corresponding to the respective operation modes.

In step S105, the CPU 110 transmits a signal for setting the imaging control unit 303 of the control PC 301 to the X-ray automatic detection mode via the entry device 317 according to the setting of the X-ray imaging apparatus 101. According to the received setting signal, the operation mode setting unit 318 of the control PC 301 sets the operation mode to the X-ray automatic detection mode. The display control unit 307 displays the set operation mode on the display 310 to notify the operator 312 of the set operation mode.

In step S106, the control PC 301 transmits the wireless LAN connection related information to the X-ray imaging apparatus 101 via short-range wireless communication of the entry device 317. The X-ray imaging apparatus 101 makes wireless LAN settings according to the received wireless LAN connection related information (communication parameters), and establishes wireless LAN communication connection with the access point 201. Thus, communication between the control PC 301 and the X-ray imaging apparatus 101 is established. The processing in step S106 is performed in response to the completion of the setting of the operation mode. According to such an operation flow, if the operation mode is not set, no communication parameter is set and the X-ray imaging apparatus 101 is controlled not to be able to connect to the wireless LAN network where the X-ray generation apparatus 402 and the control PC 301 are. As a result, if the operation mode is inappropriate, the wireless communication of the X-ray imaging apparatus 101 is disabled to perform control for prohibiting radiation imaging by the X-ray imaging apparatus 101 and the X-ray generation apparatus 402 that have mutually incompatible functions.

In step S107, the CPU 110 determines whether the X-ray manual synchronization mode can be performed. Here, the CPU 110 refers to the function information and determines whether the X-ray imaging apparatus 101 includes a corresponding program and whether the components of the X-ray imaging apparatus 101 are in an appropriate state. The CPU 110 thus determines whether the X-ray manual synchronization mode can be performed. If the CPU 110 determines that the X-ray manual synchronization mode can be performed (YES in step S107), the processing proceeds to step S108. If the CPU 110 determines that the X-ray manual synchronization mode cannot be performed (NO in step S107), the processing proceeds to step S110.

In step S108, the operation mode control circuit 113 sets the operation mode of the X-ray imaging apparatus 101 to the X-ray manual synchronization mode. The operation mode control circuit 113 stores information indicating the setting into the memory 111, and transmits the information to the drive control circuit 105. In the drive control circuit 105, circuitry for performing control corresponding to the X-ray manual synchronization mode is activated.

In step S109, the operation mode setting unit 318 sets the imaging control unit 303 of the control PC 301 to the X-ray manual synchronization mode according to the setting of the X-ray imaging apparatus 101. The display control unit 307 displays the set operation mode on the display 310 to notify the operator 312 of the set operation mode.

In step S110, the operation mode setting unit 318 transmits no command for setting the operation mode to the X-ray imaging apparatus 101. The display control unit 307 displays on the display 310 that the X-ray imaging apparatus 101 to be registered is unusable, thereby notifying the operator 312 of a warning.

In step S110, the operation mode setting unit 318 issues the warning if none of the operation modes can be set. Instead, before the operation mode setting unit 318 performs the processing in step S110, the X-ray imaging apparatus 101 may make settings to communicate with the control PC 301 as in step S106. In such a case, the warning is issued that the imaging by the X-ray imaging apparatus 101 cannot be performed after the X-ray imaging apparatus 101 and the control PC 301 has become possible to communicate with each other. As a result, even if the X-ray imaging apparatus 101 cannot operate in any of the operation modes, the X-ray imaging apparatus 101 can solve the problem or do maintenance by performing a functional diagnosis according to control by the control PC 301.

The foregoing example has dealt with the case of making wireless communication settings. The X-ray imaging apparatus 101 may include a connector instead of or in addition to the short-range wireless communication circuit 106, and set the operation mode and exchange the communication parameters by wired cable communication.

In another example, the control PC 301 may set the operation mode instead of the X-ray imaging apparatus 101, and transmit a signal for setting the operation mode to the X-ray imaging apparatus 101 via the entry device 317. In such a case, the processing of steps S102, S103, S104, S107, and S108 is modified like the following steps S102′, S103′, S104′, S107′, and S108′.

In step S102′, the X-ray imaging apparatus 101 transmits the function information (information about the operation mode(s) supported by the X-ray imaging apparatus 101) recorded in the function information memory 114 to the entry device 317 via the short-range wireless communication circuit 106.

The entry device 317 transmits the received function information to the operation mode setting unit 318 of the control PC 301.

In step S103′, the operation mode setting unit 318 determines based on the received function information whether the X-ray imaging apparatus 101 that the operator 312 is attempting to register has an X-ray automatic detection function.

If the operation mode setting unit 318 determines that the X-ray imaging apparatus 101 has the X-ray automatic detection function (YES in step S103′), the processing proceeds to step S104′. If the operation mode setting unit 318 determines that the X-ray imaging apparatus 101 does not have the X-ray automatic detection function (NO in step S103′), the processing proceeds to step S107′.

In step S104′, the operation mode setting unit 318 transmits, via the entry device 317, a command for setting the operation mode of the X-ray imaging apparatus 101 to the X-ray automatic detection mode.

Based on the received command for setting the operation mode, the X-ray imaging apparatus 101 sets the operation mode of the operation mode control circuit 113 to the X-ray automatic detection mode.

In step S107′, the operation mode setting unit 318 determines based on the received function information whether the X-ray imaging apparatus 101 that the operator 312 is attempting to register has an X-ray manual synchronization function.

If the operation mode setting unit 318 determines that the X-ray imaging apparatus 101 has the X-ray manual synchronization function (YES in step S107′), the processing proceeds to step S108′. If the operation mode setting unit 318 determines that the X-ray imaging apparatus 101 does not have the X-ray manual synchronization function (NO in step S107′), the processing proceeds to step S110′.

In step S108′, the operation mode setting unit 318 transmits, via the entry device 317, a command for setting the operation mode of the X-ray imaging apparatus 101 to the X-ray manual synchronization mode.

Based on the received command for setting the operation mode, the X-ray imaging apparatus 101 sets the operation mode of the operation mode control circuit 113 to the X-ray manual synchronization mode.

Suppose, for example, that an X-ray imaging apparatus having only an X-ray synchronization control function (X-ray synchronization mode) is used as the X-ray imaging apparatus 101 to be registered. According to the foregoing flow, such an X-ray imaging apparatus 101 is not registered and the operator 312 is notified of a warning.

This configuration can prevent re-imaging due to the use of the X-ray imaging apparatus 101 not having the X-ray automatic detection function or the X-ray manual synchronization function in combination with the X-ray generation apparatus 402 having no function for synchronization control on the X-ray irradiation timing.

If an X-ray imaging apparatus having the X-ray manual synchronization function and the X-ray synchronization control function is used as the X-ray imaging apparatus 101 to be registered, the operation mode of the X-ray imaging apparatus 101 and the control PC 301 is automatically set to the X-ray manual synchronization mode.

If an X-ray imaging apparatus having the X-ray automatic detection function, the X-ray manual synchronization function, and the X-ray synchronization control function is used as the X-ray imaging apparatus 101 to be registered, the operation mode of the X-ray imaging apparatus 101 and the control PC 301 is automatically set to the X-ray automatic detection mode.

This configuration eliminates the need for the operator 312 to make the setting. The operator 312 can also be prevented from setting an erroneous operation mode.

A second exemplary embodiment of the present invention will be described.

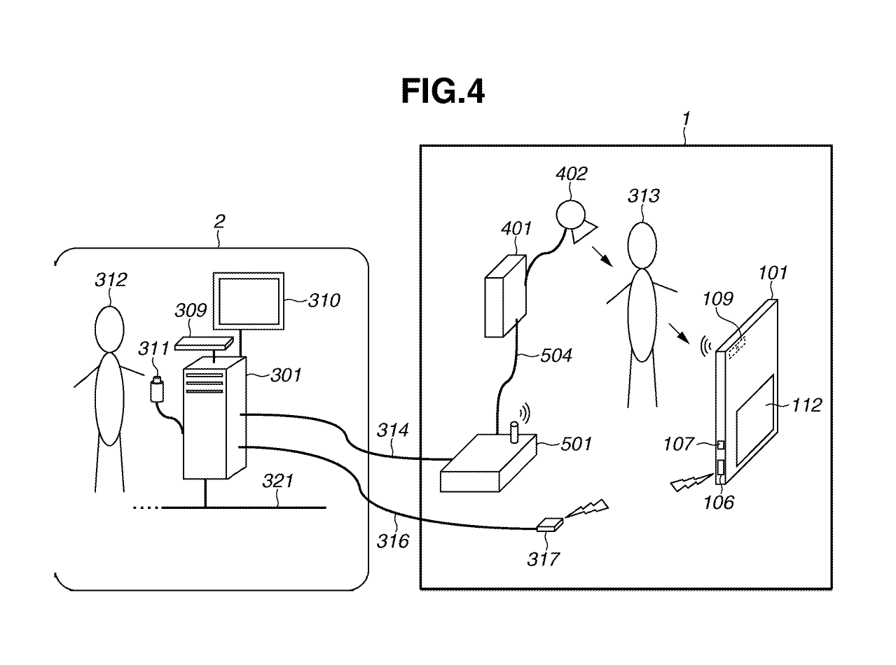

FIG. 4 is a schematic diagram illustrating an example of a schematic configuration of a wireless X-ray imaging system according to the second exemplary embodiment of the present invention.

The second exemplary embodiment deals with an X-ray imaging system that has a function for synchronization control on X-ray irradiation timing.

The configuration in FIG. 4 includes similar components to those described in FIG. 1 of the first exemplary embodiment. A description of such components will be omitted.

In FIG. 4, a synchronization access point 501 performs wireless communication with the X-ray imaging apparatus 101 and controls synchronization with the X-ray generation apparatus 402. A connection cable 504 connects the synchronization access point 501 and the X-ray control apparatus 401 in a wired manner.

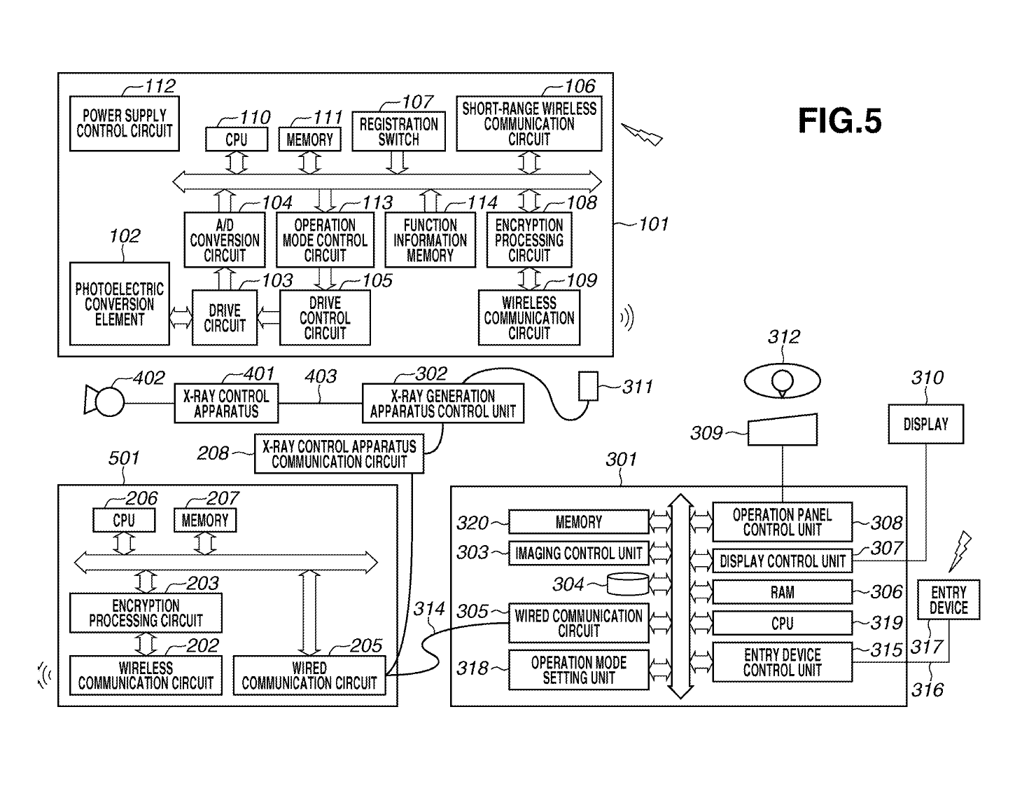

A detailed configuration of the apparatuses will be described with reference to the block diagram in FIG. 5.

A description of components in FIG. 5 similar to those described in FIG. 2 of the first exemplary embodiment will be omitted.

(FIG. 5 differs from FIG. 1 only in that the synchronization access point 501 has an additional X-ray irradiation synchronization control function, and that a connection cable of the X-ray control apparatus 401 is connected to the synchronization access point 501.)

The synchronization access point 501 includes the wireless communication circuit 202, the encryption processing circuit 203, the wired communication circuit 205, the CPU 206, the memory 207, and the X-ray control apparatus communication circuit 208.

The X-ray control apparatus communication circuit 208 communicates with the X-ray control apparatus 401 and performs synchronization control on X-ray irradiation, based on instructions from the X-ray generation apparatus control unit 302 of the control PC 301 and instructions from the X-ray imaging apparatus 101.

A procedure for setting the operation mode of the X-ray imaging apparatus 101 and the control PC 301 according to the second exemplary embodiment of the present invention will be described in detail.

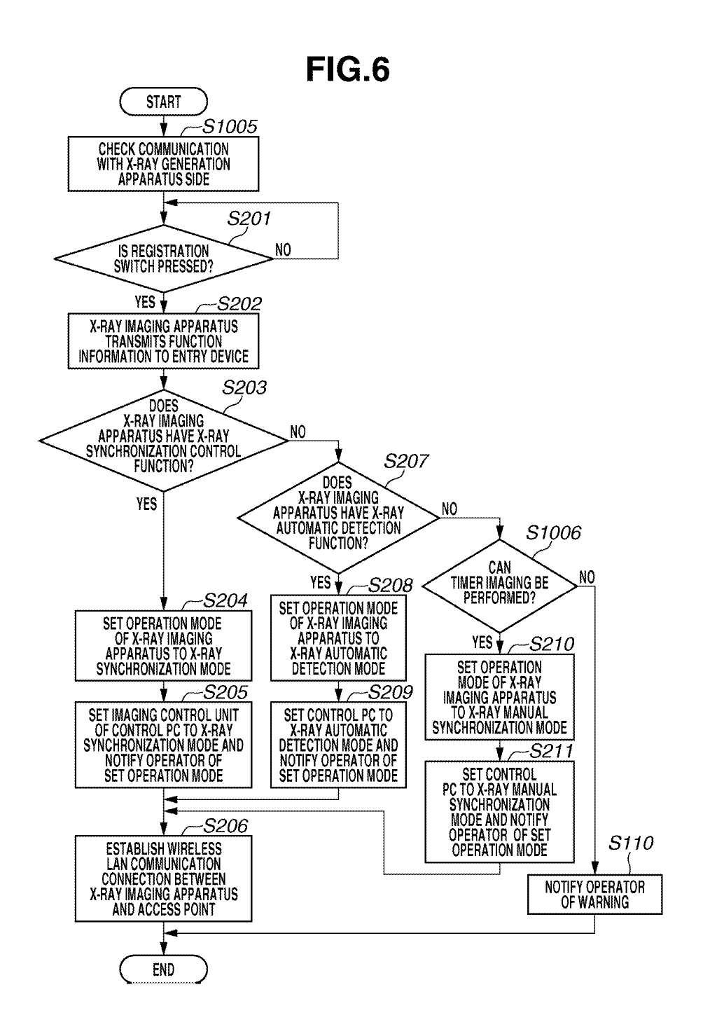

FIG. 6 is a flowchart illustrating an operation when registering the X-ray imaging apparatus 101 with the X-ray imaging system. In step S1005, the control PC 301 checks communication with the X-ray generation 402 side, as explained in the above-described embodiment.

In step S201, the CPU 110 detects pressing of the registration switch 107 of the X-ray imaging apparatus 101. If the CPU 110 detects that the registration switch 107 is pressed (YES in step S201), the processing proceeds to step S202. (The CPU 110 waits until the registration switch 107 is pressed.)

In step S202, the X-ray imaging apparatus 101 transmits the function information (information about the operation mode(s) supported by the X-ray imaging apparatus 101) recorded in the function information memory 114 to the entry device 317 via the short-range wireless communication circuit 106.

The entry device 317 transmits the received function information to the operation mode setting unit 318 of the control PC 301.

In step S203, the operation mode setting unit 318 determines based on the received function information whether the X-ray imaging apparatus 101 that the operator 312 is attempting to register has the X-ray synchronization control function.

If the operation mode setting unit 318 determines that the X-ray imaging apparatus 101 has the X-ray synchronization control function (YES in step S203), the processing proceeds to step S204. If the operation mode setting unit 318 determines that the X-ray imaging apparatus 101 does not have the X-ray synchronization control function (NO in step S203), the processing proceeds to step S207.

In step S204, the operation mode setting unit 318 transmits, via the entry device 317, a command for setting the operation mode of the X-ray imaging apparatus 101 to the X-ray synchronization mode.

Based on the received command for setting the operation mode, the X-ray imaging apparatus 101 sets the operation mode of the operation mode control circuit 113 to the X-ray synchronization mode.

In step S205, the operation mode setting unit 318 sets the imaging control unit 303 of the control PC 301 to the X-ray synchronization mode according to the setting of the X-ray imaging apparatus 101. The display control unit 307 displays the set operation mode on the display 310 to notify the operator 312 of the set operation mode.

In step S206, the control PC 301 transmits the wireless LAN connection related information to the X-ray imaging apparatus 101 via short-range wireless communication of the entry device 317.

The X-ray imaging apparatus 101 makes wireless LAN settings according to the received wireless LAN connection related information, and establishes wireless LAN communication connection with the access point 201.

In step S207, the operation mode setting unit 318 determines based on the received function information whether the X-ray imaging apparatus 101 that the operator 312 is attempting to register has the X-ray automatic detection function.

If the operation mode setting unit 318 determines that the X-ray imaging apparatus 101 has the X-ray automatic detection function (YES in step S207), the processing proceeds to step S208. If the operation mode setting unit 318 determines that the X-ray imaging apparatus 101 does not have the X-ray automatic detection function (NO in step S207), the processing proceeds to step S1006. In step S1006, the operation mode setting unit 318 determines whether the timer imaging mode can be performed. If operation mode setting unit 318 determines that the timer imaging can be performed (Yes in step S1006), the processing proceeds to step S210. If operation mode setting unit 318 determines that the timer imaging cannot be performed (NO in step S1006), in step S110, the operation mode setting unit 318 transmits no command for setting the operation mode to the X-ray imaging apparatus 101. The display control unit 307 displays on the display 310 that the X-ray imaging apparatus 101 to be registered is unusable, thereby notifying the operator 312 of a warning. The processing then ends.

In step S208, the operation mode setting unit 318 transmits a command for setting the operation mode of the X-ray imaging apparatus 101 to the X-ray automatic detection mode via the entry device 317.

Based on the received command for setting the operation mode, the X-ray imaging apparatus 101 sets the operation mode of the operation mode control circuit 113 to the X-ray automatic detection mode.

In step S209, the operation mode setting unit 318 sets the imaging control unit 303 of the control PC 301 to the X-ray automatic detection mode according to the setting of the X-ray imaging apparatus 101. The display control unit 307 displays the set operation mode on the display 310 to notify the operator 312 of the set operation mode.

In step S210, the operation mode setting unit 318 transmits, via the entry device 317, a command for setting the operation mode of the X-ray imaging apparatus 101 to the X-ray manual synchronization mode.

Based on the received command for setting the operation mode, the X-ray imaging apparatus 101 sets the operation mode of the operation mode control circuit 113 to the X-ray manual synchronization mode.

In step S211, the operation mode setting unit 318 sets the imaging control unit 303 of the control PC 301 to the X-ray manual synchronization mode according to the setting of the X-ray imaging apparatus 101. The display control unit 307 displays the set operation mode on the display 310 to notify the operator 312 of the set operation mode.

The procedure for setting the operation mode of the X-ray imaging apparatus 101 and the control PC 301 according to the second exemplary embodiment of the present invention has been described above.

Suppose, for example, that an X-ray imaging apparatus having the X-ray synchronization control function is used as the X-ray imaging apparatus 101 to be registered in the X-ray imaging system having the function for synchronization control on the X-ray irradiation timing. According to the foregoing flow, such an X-ray imaging apparatus 101 is automatically set to the X-ray synchronization mode. This configuration eliminates the need for the operator 312 to make the setting. The operator 312 can also be prevented from setting an erroneous operation mode.

The X-ray automatic detection function does not always function well. It may be difficult to detect the X-ray irradiation depending on various conditions including the irradiation condition of the X-rays, the size of the irradiation area, the physique and irradiation region of the patient 313, and the X-ray start-up characteristic of each X-ray tube. If the X-ray imaging system has the X-ray synchronization control function and the X-ray imaging apparatus 101 has the X-ray synchronization control function as well, the X-ray synchronization mode may therefore be used with priority. This configuration can avoid the risk of failing to automatically detect X-rays depending on the imaging condition.

With the X-ray manual synchronization function, the operator 312 needs to adjust the X-ray irradiation timing. In such a case, the X-ray synchronization mode can also be used with priority.

Consequently, in the second exemplary embodiment, the X-ray imaging apparatus 101 having the X-ray synchronization control function sets the X-ray synchronization mode with the highest priority.

The first and second exemplary embodiments have dealt with the case of setting the X-ray automatic detection mode with priority over the X-ray manual synchronization mode. However, higher priority may be given to the X-ray manual synchronization mode over the X-ray automatic detection mode. The X-ray imaging system may be configured so that which mode to select is set by the operator 312.

The communication unit used in registering the X-ray imaging apparatus 101 has been described by using short-range wireless communication as an example. However, the communication unit is not limited to this example. For example, a cradle or charger which holds the X-ray imaging apparatus 101 may include a communication unit, which can be used to perform registration. Alternatively, a cable may be used to perform registration by a wired connection.

The second exemplary embodiment has dealt with the case where the synchronization access point 501 and an X-ray synchronization control apparatus are integrally configured. However, the synchronization access point 501 and the X-ray synchronization control apparatus may be separately configured.

According to the first and second exemplary embodiments, re-imaging due to a combination of the functions of the X-ray imaging apparatus 101 and the X-ray generation apparatus 402 can be prevented. In addition, troublesome settings can be eliminated to avoid erroneous settings.

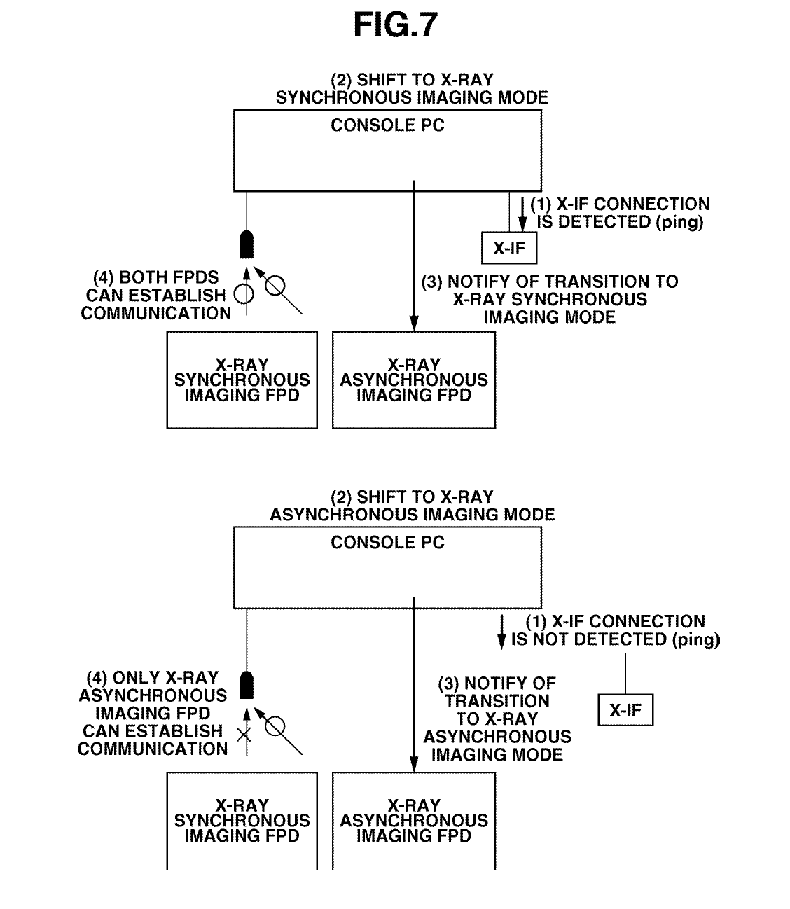

Referring to FIG. 7, an example of an operation setting according to the exemplary embodiments will be described. In (1), a console PC (control PC 301) initially checks by using a ping command whether an X-interface (X-IF; X-ray control apparatus communication circuit 208) is connected to the synchronization access point 501. If the connection of the X-IF is confirmed, then in (2), the console PC shifts to an X-ray synchronous imaging mode (X-ray synchronization mode). From the viewpoint of reliability, a flat panel detector (FPD; X-ray imaging device 101) normally supports the X-ray synchronization mode. When the X-IF is connected, the console PC is thus controlled to operate in the X-ray synchronization mode. Next, in (3), the console PC transmits to a FPD an instruction signal for causing the FPD to operate in the X-ray synchronization mode via the entry device 317, for example. Under such circumstances, in (4), an FPD that only has the X-ray synchronization mode and an FPD that has the X-ray automatic detection mode and also the X-ray manual synchronization mode can both establish communication with the console PC by the control of the console PC.

If, in (1), the connection of the X-IF is not confirmed, then in (2), the console PC immediately shifts to an X-ray asynchronous imaging mode. The X-ray asynchronous imaging mode is an operation mode of the console PC and FPDs for performing the X-ray automatic detection mode and the X-ray manual synchronization mode. In such a case, in (3), the console PC transmits a signal for instructing to shift to the X-ray asynchronous imaging mode to the FPDs that are transmitting a wireless LAN connection request. Under such circumstances, in (4), the FPD having the X-ray asynchronous imaging mode can establish wireless LAN connection via the entry device 317. The FPD having only the X-ray synchronization mode cannot establish wireless LAN connection.

In such a manner, the console PC (control PC 301) detects the connection of the X-IF and sets the operation mode on the console PC side before the FPDs establish wireless connection. The console PC then sets the operation mode of the FPDs requesting wireless connection. As a result, radiation imaging in an appropriate imaging mode can be efficiently performed. Since the console PC performs control to reject a connection request depending on the situation, problems such as erroneous irradiation of radiations can be reduced.

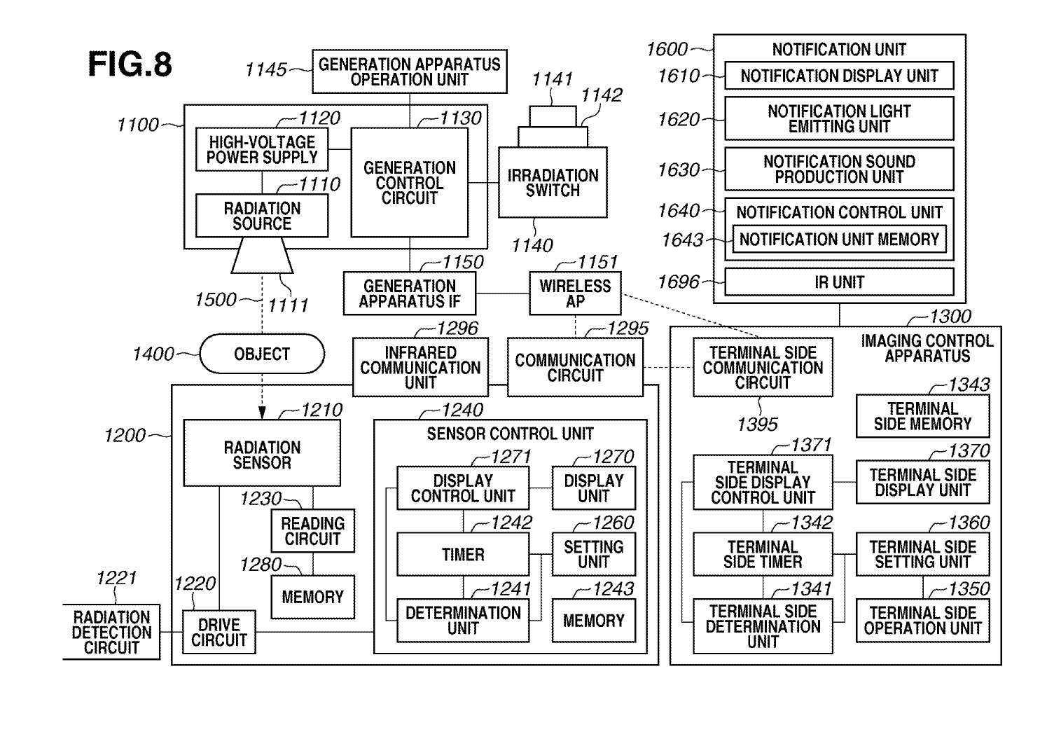

A configuration of a radiation imaging system according to another exemplary embodiment will be described with reference to FIG. 8.

The radiation imaging system includes a radiation generation apparatus 1100. The radiation generation apparatus 1100 includes a radiation source 1110, a radiation diaphragm 1111, a high-voltage power supply 1120, a generation control circuit 1130, a generation apparatus operation unit 1145, and an irradiation switch 1140. For example, the radiation source 1110 includes a reflective or transmissive target and an electron source that generates electrons. The radiation source 1110 causes electrons to impinge on the target to generate radiations. The radiation diaphragm 1111 includes a plurality of radiation shielding members. The shielding members are appropriately arranged to shape a radiation flux into a desired shape. For example, a radiation flux is shaped to have a rectangular or circular cross section perpendicular to the irradiation direction. The high-voltage power supply 1120 generates voltage for accelerating the electrons generated by the electron source. The generation control circuit 1130 controls the radiation generation.

The generation apparatus operation unit 1145 is an operation unit for setting a generation condition of the radiations to be generated. For example, the generation condition is input in a form such as a tube current, a tube voltage, and a milliampere-second (mAs) value. The generation control circuit 1130 receives the input generation condition and controls an operation according to the generation condition. The irradiation switch 1140 is a switch for controlling the generation timing of the radiations. A first switch 1141 in the first stage is pressed to input a rotor up signal to the generation control circuit 1130. The generation control circuit 1130 starts the rotation of an anode of the radiation source 1110. After the completion of the rotor-up, a second switch 1142 in the second stage is pressed to input a radiation signal to the generation control circuit 1130, whereby the radiation generation is started. A transmissive target needs no rotor-up, whereas the control flow including the preparation operation by using the first switch 1141 and the start of exposure by using the second switch 1142 need not be changed. With a transmissive target, the switch may be a single-stage one.

The radiations generated by the foregoing radiation generation apparatus 1100 are detected by an radiation sensor 1210 which is appropriately positioned. A radiation photographing system (a radiation imaging apparatus 1200) will be described below.

The radiation imaging apparatus 1200 includes the radiation sensor 1210, a drive circuit 1220, a reading circuit 1230, a sensor control unit 1240, a determination unit 1241, a timer 1242, a setting unit 1260 (time setting unit), a display unit 1270, a display control unit 1271, and a memory 1280. Such components can exchange signals via a wired or wireless network and/or by electrical connection. The sensor control unit 1240, the determination unit 1241, the timer 1242, an operation unit, the setting unit 1260, and the display control unit 1271 control the present radiation imaging system. The radiation sensor 1210, the drive circuit 1230, the sensor control unit 1240, and the memory 1280 are included in a radiation imaging unit. The sensor control unit 1240, the determination unit 1241, the setting unit 1260, and the display control unit 1271 are implemented, for example, by an FPGA. To provide the functions and processing of such components, configuration data about the logic circuits on the FPGA is generated by using a hardware description language. The FPGA is then configured by using the configuration data.

The radiation imaging apparatus 1200 is of a portable type. The radiation sensor 1210, the drive circuit 1220, the reading circuit 1230, the sensor control unit 1240, the memory 1280, and a battery 1290 are mounted in a casing of the radiation imaging apparatus 1200. The sensor control unit 1240 includes the display control unit 1271, the timer 1242, the determination unit 1241, and the setting unit 1260 aside from the foregoing imaging control functions. The sensor control unit 1240 is implemented, for example, by one or a plurality of FPGAs. The battery 1290 is a power supply for supplying power to the components in the radiation imaging apparatus 1200.

The radiation sensor 1210 includes a two-dimensional array of a plurality of pixels which accumulate charge according to reception of radiations. The radiation sensor 1210 has an accumulation mode for accumulating charge and a reading mode for reading an electrical signal based on the accumulated charge. For example, the radiation sensor 1210 includes row selection lines which are connected to the drive circuit 1220 row by row, and column signal lines which are connected to the reading circuit 1230 column by column. Each pixel includes a positive intrinsic negative (PIN) or metal insulator semiconductor (MIS) type photoelectric conversion element and a thin film transistor (TFT) which connects the photoelectric conversion element with a column signal line. A base side electrode of the TFT is connected with a row selection line and controlled to be on/off by the drive circuit 1220. The TFT is turned on to shift to an accumulation state or the accumulation mode for accumulating charge in the pixel. The TFT is turned off to shift to a reading state or the reading mode for reading the accumulated charge.

The timing when the radiation sensor 1210 shifts to the accumulation mode from the reading mode is controlled by the operation unit, the setting unit 1260, the timer 1242, the determination unit 1241, and the sensor control unit 1240. The operation unit includes a hardware button and/or a software button displayed on a display screen of the display unit 1270. The operation unit receives, from the user, a time input for setting a wait time and an instruction input for starting an imaging operation. The setting unit 1260 stores information about the input time into a memory 1243 as information about the wait time. According to the instruction input via the operation unit, the radiation imaging system starts measuring the wait time.

The timer 1242 includes, for example, a clock pulse generator and a counter circuit. The counter circuit repeats counting up at regular intervals by using clock pulses and outputs a count value. The determination unit 1241 monitors the timer 1242 and repeats determination processing for determining whether the wait time has elapsed from when a start input is made. The timer 1242 and the determination unit 1241 can thus determine the lapse of the wait time input by the user.

After the lapse of the wait time, the sensor control unit 1240 performs control, for example, to transmit a control signal for instructing the drive circuit 1220 to change a mode so that the radiation sensor 1210 shifts to the accumulation mode. As a result, the radiation sensor 1210 can shift to the accumulation state after a wait for a desired time input by the user.

In the accumulation mode, the user performs X-ray irradiation for imaging according to the timing when the radiation sensor 1210 starts an accumulation operation. The user is notified of the timing when the X-ray irradiation can be performed, and performs the irradiation according to the notification. In such a manner, the irradiation timing of the X-rays and the imaging timing can be synchronized for imaging.

Since the user-changeable wait time is provided before the transition to the accumulation mode, the start timing of the accumulation mode can be adjusted to user-desired timing. For example, if the radiation imaging may be immediately started, the wait time can be set to three seconds or less, or one second or less, to immediately shift the radiation sensor 1210 to the accumulation state to start imaging. The wait time can even be set to zero seconds to immediately shift the radiation sensor 1210 to the accumulation state according to the instruction without a wait. On the other hand, if the transition to the accumulation mode needs to be timed with a subject or if enough time is needed, the wait time may be set to 10 seconds or so.

The user presses the irradiation switch 1140 according to the transition to the accumulation state, thereby causing the radiation generation apparatus 1100 to generate radiations. Then, the radiation sensor 1210 in the accumulation state can accumulate charge according to the intensity of the radiations. After a lapse of a predetermined fixed accumulation time, for example, the sensor control unit 1240 outputs an instruction to the drive circuit 1220 to cause the radiation sensor 1210 to shift to the reading mode according to the end of the accumulation period. The electrical signal read by the reading circuit 1230 is amplified and A/D-converted to generate digital radiation image data. The memory 1280 stores the digital radiation image data. The memory 1280 stores various imaging conditions and setting values aside from the digital radiation image data. An image check terminal includes, for example, a communication unit capable of wired and wireless communication, a display control unit, and a display unit. The communication unit receives the digital radiation image data. The display control unit causes the display unit to display the digital radiation image data. The digital radiation image data can also be transmitted and stored into an external server by wired or wireless communication via the communication unit.

The radiation imaging apparatus 1200 may include the display unit 1270 and the display control unit 1271. For example, the display control unit 1271 causes the display unit 1270 to display that it is a period in which the radiation irradiation should be avoided, from when the instruction is input until the wait time elapses. In such a manner, the display unit 1270 can inform the user of the period in which charge should not be accumulated. In another example, the display control unit 1271 causes the display unit 1270 to display that irradiation should be started, according to the lapse of the wait time or the transition to the accumulation mode. In such a manner, the display unit 1270 can inform the user of the timing for imaging.

The display control unit 1271 further causes the display unit 1270 to provide a display according to the remaining time until the time to perform the irradiation of radiations. This configuration greatly facilitates the user finding out the timing to press the irradiation switch 1140. Examples of the display according to the remaining time until the lapse of the wait time include a countdown display of the remaining time on the display unit 1270 and blinking of a light-emitting diode (LED) in gradually decreasing intervals.

The radiation imaging system according to the exemplary embodiment may further include a generation apparatus interface (IF) 1150 which electrically connects the radiation photographing system and the radiation generation apparatus 1100. The generation apparatus IF 1150 is a unit intended to transfer/receive synchronization signals for synchronizing the generation of radiations with the accumulation mode to/from the radiation generation apparatus 1100. If the radiation generation apparatus 1100 includes an interface for outputting the generation timing of radiations, the interfaces can be connected for synchronization. In an example of exchange of the synchronization signals, the generation control circuit 1130 outputs a signal requesting a permission for radiation exposure if the first switch 1141 has been pressed to complete the rotor-up and then the second switch 1142 is pressed. The generation apparatus IF 1150 receives the signal. The sensor control unit 1240 in response causes the radiation sensor 1210 to shift to the accumulation mode. The sensor control unit 1240 may perform control to read charge accumulated in the radiation sensor 1210 and/or other predetermined initialization processing. According to the transition to the accumulation mode, the sensor control unit 1240 transmits a radiation enabling signal via the generation apparatus IF 1150. The generation control circuit 1130 receives the radiation enabling signal and causes the radiation source 1110 to generate radiations. Such synchronization control is performed to perform the radiation imaging using the radiation sensor 1210 without fail by the operation of pressing the irradiation switch 1140, which is similar to that performed with a conventional analog film. Note that the radiation generation apparatus 1100 may not have such an interface due to a difference between the manufacturers of the radiation generation apparatus 1100 and the radiation photographing system. The radiation generation apparatus 1100 may have no such interface depending on the model. In such cases, the user can easily perform radiation imaging using the radiation sensor 1210 by matching the irradiation timing by the foregoing timer control and display control.

The radiation photographing system according to the exemplary embodiment may further include a radiation detection circuit 1221. The radiation detection circuit 1221 is used to detect radiation irradiation from the radiation generation apparatus 1100 and obtain an image if the radiation generation apparatus 1100 cannot be synchronized as described above. For example, the radiation detection circuit 1221 monitors the pixels of the radiation sensor 1210 and monitors current output from the pixels to detect the generation of radiations. Alternatively, a dedicated sensor using a semiconductor element having sensitivity to radiations may be provided aside from the radiation sensor 1210. The dedicated sensor may be arranged on the front surface, the outer periphery, or the rear surface of the radiation sensor 1210. The radiation irradiation can be detected according to the output of such a sensor. In another example, the radiation sensor 1210 is in the reading mode before the radiation irradiation is detected. In response to the detection of the radiation irradiation, the radiation sensor 1210 shifts to the accumulation mode to obtain a radiation image. In yet another example, the radiation sensor 1210 repeats accumulation and reading from before the detection of the radiation irradiation. The radiation sensor 1210 can generate radiation image data by using the data read in a period during which the irradiation of radiations is performed.

Such a radiation detection circuit 1221 can be used to control the radiation sensor 1210. Special imaging, for example, imaging with an extremely low dose, however, may fail to be appropriately performed due to a delay in detecting radiations. Moreover, when using the radiation detection circuit 1221, the radiation sensor 1210 needs to be operated by a predetermined drive before the detection of radiations.

Imaging control using the timer 1242 (first control, the X-ray manual synchronization mode), imaging control using the radiation detection circuit 1221 (second control, the X-ray automatic detection mode), and synchronous imaging control using the generation apparatus IF 1150 (third control, the X-ray synchronization mode) have been described above. Among such control, which imaging mode (operation mode) to be used may be selected as appropriate according to the system configuration, an imaging environment, and the imaging condition. For example, the setting unit 1260 switches the setting of the imaging mode according to an operation from the operation unit or an external signal. To perform synchronous imaging, the setting unit 1260 stores an imaging mode setting value of 0 in the memory 1243. When the radiation detection unit 1221 is used, the setting unit 1260 stores an imaging mode setting value of 1 in the memory 1243. When imaging is performed using the timer 1242, the setting unit 1260 stores an imaging mode setting value of 2 in the memory 1243. The determination unit 1241 refers to the setting value as appropriate and causes the radiation imaging apparatus 1200 to operate in the set mode at the time of imaging.

A communication circuit 1295 is a communication circuit for communicating with a wireless access point (AP) 1151 or a terminal side communication circuit 1395. An infrared communication unit 1296 is an example of a short-range wireless communication unit. The infrared communication unit 1296 communicates with an infrared (IR) unit 1696 of a notification unit 1600 (corresponding to the entry device 317) which is connected on a terminal side.

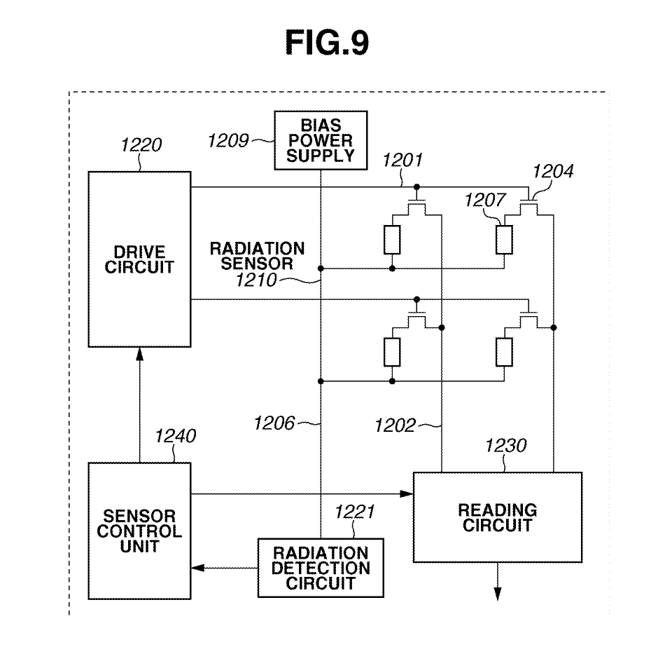

An imaging control apparatus 1300 different from the portable radiation imaging apparatus 1200 includes a component corresponding to the timer 1242. The imaging control apparatus 1300 functions as a control apparatus of the radiation imaging system as a modality. The imaging control apparatus 1300 is an apparatus different from the radiation imaging apparatus 1200 and mounted in an independent casing. The imaging control apparatus 1300 operates while wirelessly communicating with the radiation imaging apparatus 1200 without a physical connection. In the example in FIG. 8, the imaging control apparatus 1300 has functions as the control apparatus of the radiation imaging apparatus 1200. However, the function of the imaging control apparatus 1300 is not limited to the above. The imaging control apparatus 1300 may have functions as a control apparatus that sets the irradiation condition of the radiation generation apparatus 1100. The imaging control apparatus 1300 is connected to an in-house network to receive order information about radiation imaging from a radiology information system (RIS), and to transmit captured images to picture archiving and communication systems (PACSs). The imaging control apparatus 1300 includes, for example, a CPU, a RAM, a read-only memory (ROM), a hard disk drive (HDD), and a communication circuit. The ROM or HDD stores a computer program including instructions for implementing processing described below with reference to the flowchart in FIG. 9. The CPU reads and executes the program as appropriate to implement the functions of the imaging control apparatus 1300.

The imaging control apparatus 1300 includes a terminal side determination unit 1341, a terminal side timer 1342, a terminal side operation unit 1350, a terminal side setting unit 1360 (time setting unit), a terminal side display unit 1370, and a terminal side display control unit 1371, which have functions similar to, except for a few, those of the determination unit 1241, the timer 1242, the setting unit 1260, the display unit 1270, and the display control unit 1271 illustrated in FIG. 8.

The terminal side operation unit 1350 includes a hardware button and/or a software button displayed on a display screen of the terminal side display unit 1370. The terminal side operation unit 1350 receives, from the user, a time input for setting a wait time and an instruction input for starting an imaging operation. The terminal side setting unit 1360 stores information about the input time into a terminal side memory 1343 as information about the wait time. In response to the instruction input via the terminal side operation unit 1350, the radiation imaging system starts measuring the wait time.

The notification unit 1600 is an example of an indicator related to the radiation imaging using the radiation sensor 1210. For example, the radiation imaging apparatus 1200 may be arranged on the back side of the subject lying on a bed. In such a case, the display unit 1270 arranged on a side surface of the casing of the radiation imaging apparatus 1200 can be hidden under the subject. The notification unit 1600 is provided to notify the operator of the state of the radiation imaging apparatus 1200 even in such a case. The notification unit 1600 includes a notification display unit 1610, a notification light emitting unit 1620, a notification sound production unit 1630, a notification control unit 1640, and the IR unit 1696. The notification display unit 1610 displays information such as a character string and an icon by using, for example, a liquid crystal display. The notification light emitting unit 1620 includes an LED and notifies the operator of the state of the radiation imaging apparatus 1200 by using a light emission pattern. The notification sound production unit 1630 produces sound from a speaker. Such units are controlled by the notification control unit 1640. The notification control unit 1640 receives a first signal and a second signal in a wired or wireless manner. The first signal indicates a start of measuring of the wait time. The second signal indicates the lapse of the wait time from the start of the measuring. Before the wait time set by the setting unit 1260 is determined to have elapsed, the notification control unit 1640 causes the notification unit 1600 to issue a notification according to the remaining time until the lapse of the wait time. If the wait time is determined to have elapsed, the notification control unit 1640 causes the notification unit 1600 to issue a notification indicating that irradiation should be started. Using such a separate notification apparatus, the operator can find out the timing to perform irradiation even in a position away from the radiation imaging apparatus 1200. The IR unit 1696 is an example of the short-range wireless communication circuit according to the first exemplary embodiment. The notification unit 1600 also functions as the entry device 317.

A configuration example of the radiation sensor 1210 and its accompanying circuits will be described with reference to FIG. 9. For the sake of simplicity, FIG. 9 illustrates a solid image sensor of the radiation sensor 1210 including a plurality of pixels arranged in a 2×2 two-dimensional matrix (two-dimensional sensor) and its accompanying circuits. In fact, a solid image sensor including several thousand rows×several thousand columns of pixels is used as an X-ray sensor unit. The numbers of rows and columns of the pixels and the number of pixels are not limited in particular. In an exemplary embodiment, a phosphor for converting radiations into visible light is stacked on the solid image sensor. In another example, the solid image sensor itself converts radiations into an electrical signal.

Each pixel of the radiation sensor 1210 includes a photoelectric conversion element 1207 and a TFT 1204 connected to one end of the photoelectric conversion element 1207. The other end of the photoelectric conversion element 1207 is connected to a bias power supply 1209 via a bias line 1206. The TFT 1204 functions as a switch element for switching between connection and disconnection of the photoelectric conversion element 1207 to and from a column signal line 1202. A base-side electrode of the TFT 1204 is connected to a row selection line 1201. The TFTs 1204 are controlled by the drive circuit 1220 row by row via respective shared row selection lines 1201. For example, the drive circuit 1220 includes a shift register which outputs signals sequentially in one direction. The drive circuit 1220 sequentially applies voltage to one of the row selection lines 1201 according to an input clock pulse. The TFTs 1204 connected to the voltage-applied row selection line 1201 are turned on accordingly. The operation of turning on the TFTs 1204 by the application of the voltage will be referred to as row selection. Sequentially performing row selection will be referred to as scanning of the radiation sensor 1210. In the exemplary embodiment illustrated in FIG. 9, the foregoing reading mode or reading state refers to a state where the scanning is sequentially performed. In the exemplary embodiment illustrated in FIG. 9, the foregoing accumulation mode or accumulation state refers to a state where all the TFTs 1204 used to generate an image are turned off.

Aside from a charge coupled device (CCD), various elements using amorphous silicon and polysilicon are known as photoelectric conversion elements. Such elements are known to accumulate charge of dark current even in a non-irradiated state regardless of the photoelectric conversion method. The charge of the dark current can cause noise during imaging of weak signals in particular. The charge of the dark current not only degrades the image quality but also lowers the sensitivity of the photoelectric conversion elements. To eliminate the charge of the dark current accumulated in the photoelectric conversion elements, a regular reset operation is needed even in a no-signal state. During the reset operation, the photoelectric conversion elements cannot accumulate charge. A desired image therefore cannot be obtained even if X-ray irradiation is performed. The resent operation is performed, for example, in the reading mode by using the TFTs 1204. In another example, reset-specific TFTs may be connected to the photoelectric conversion elements aside from the TFTs 1204.

The electrical signals read from the radiation sensor 1210 via the column signal lines 1202 are input to the reading circuit 1230. The reading circuit 1230 includes amplifiers and analog-to-digital (AD) converters, for example. The reading circuit 1230 amplifies and converts the read analog electrical signals into digital values to obtain digital radiation image data. In fact, the digital radiation image data corresponds to both charge that the photoelectric conversion elements 1207 obtains according to the radiation irradiation and dark charge that the photoelectric conversion elements 1207 produces without light reception. A dark current correction circuit for correcting dark current data components corresponding to the dark charge is thus provided. A gain correction circuit for correcting variations in the sensitivity of the pixels and a defective pixel correction circuit for correcting defective pixels may also be provided.

An example of driving using the radiation detection circuit 1221 will be described. The sensor control unit 1240 drives the drive circuit 1220 to perform scanning so that the TFTs 204 are turned on for a certain period row by row or in units of a plurality of rows. The drive circuit 1220 supplies a pulse signal to the TFTs 1204 to turn on the TFTs 1204 for a certain period. The order of scanning and the number of rows of the TFTs 1204 to be simultaneously turned on are not limited in particular.

Under the control of the sensor control unit 1240, the drive circuit 1220 performs scanning until the X-ray irradiation is detected. If all the scanning lines (row selection lines) have been driven, the radiation sensor 1210 repeats driving the scanning lines again from the first-driven one(s).

While the scanning lines are being driven, the radiation detection circuit 1221 converts the current flowing through the bias line 1206 connected to the bias power supply 1209 into a digital value through a current-voltage conversion circuit, an amplifier, an AD converter, and a signal processing circuit. A comparator compares the digital value with a predetermined threshold, and outputs a signal indicating the comparison result to the sensor control unit 1240 as an X-ray irradiation detection signal. If the digital value exceeds the predetermined threshold, the sensor control unit 1240 can determine that the current following through the bias line 1206 has changed and the X-ray irradiation is detected. The radiation detection circuit 1221 sequentially stores the digital values into a storage circuit. The state of performing such operations will be referred to as an X-ray irradiation detection state. The bias power supply 1209 is intended to supply bias voltage to the photoelectric conversion elements 1207.

The AD converter may have arbitrary sampling frequency, and may perform sampling a plurality of times while the TFTs 1204 on a scanning line are turned on. In terms of data processing, the sampled values can be averaged to finally obtain a digital value for each row. The AD converter desirably performs correlated double sampling to calculate a difference between the digital value obtained when the TFTs 1204 are turned on and the digital value obtained when the TFTs 1204 are turned off in a state where a scanning line is selected. The reason is that resistance to extraneous noise can be improved. The digital value is sequentially updated in synchronization with the scanning. The storage circuit desirably has capacity such that the digital value can be sequentially overwritten and updated and at least one digital value can be retained for all the scanning lines.

When the radiation source 1110 irradiates the radiation sensor 1210 with X-rays, a not-illustrated phosphor layer emits light which generates charge in the photoelectric conversion elements 1207. The charge flows to the bias line 1206. The flow of the charge changes the current flowing through the bias line 1206. The radiation detection circuit 1221 detects the change in the current via the foregoing circuits (current-voltage conversion circuit, amplifier, AD converter, and signal processing circuit), and outputs an instruction to stop the scanning to the sensor control unit 1240. Consequently, the radiation sensor 1210 shifts to a current accumulation state because of the X-ray irradiation. When the scanning is stopped, the storage circuit stops updating the digital value and retains the digital value. The sensor control unit 1240 stores a scanning line number (scanning line position information) for identifying the scanning line at which the scanning is stopped into a not-illustrated register. The scanning line number need not always be used if the stopped position of the scanning is otherwise identifiable.

Based on instructions from the sensor control unit 1240, the drive circuit 1220 controls the radiation sensor 1210 to shift into various operation states including resetting, radiation detection driving, charge accumulation, and reading. The operation of accumulating charge is equivalent to the operation of capturing an X-ray image of an object, thus the operation will hereinafter be referred to as an imaging operation.

The present exemplary embodiment has dealt with the case where the current flowing through the bias line 1206 is used to detect the X-ray irradiation. However, the current flowing through the bias line 1206 need not necessarily be used if current flowing inside the radiation imaging apparatus 1200 is used and the value of the current varies depending on whether the X-ray irradiation is detected.

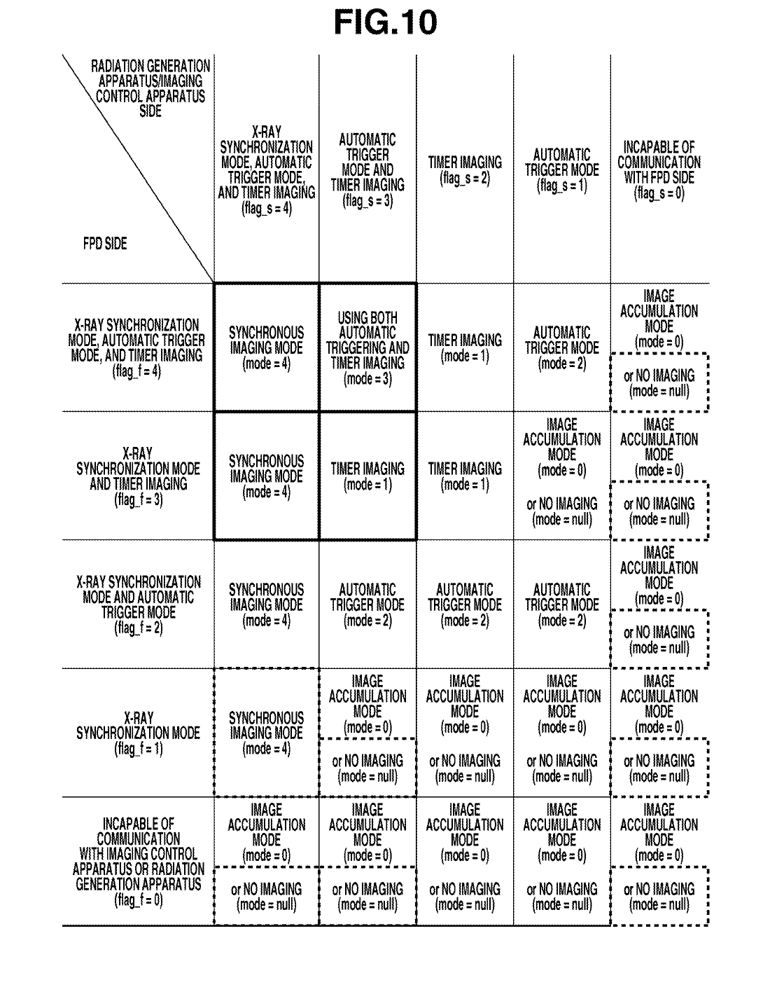

An example of the structure of setting data used to set an imaging mode (operation mode) based on function information and communication information will be described with reference to FIG. 10. The setting data is table information where combinations of function information about the radiation imaging apparatus 1200 and communication information about communication between the radiation imaging apparatus 1200 and the radiation generation apparatus 1100 is associated with the operation modes of the radiation imaging apparatus 1200. The table information is stored on the radiation imaging apparatus side or the imaging control apparatus side and used as appropriate to set the operation mode.

Function information about the FPD side (radiation imaging apparatus side) is stored in the memory 1243 as an integer parameter flag_f. If the parameter flag_f has a value of 4, it indicates that the radiation imaging apparatus 1200 has all the functions of the X-ray synchronization mode, automatic trigger mode (X-ray automatic detection mode), and timer imaging (manual synchronization). The parameter flag_f of 3 indicates that the radiation imaging apparatus 1200 has the functions of the X-ray synchronization mode and the timer imaging. The parameter flag_f of 2 indicates that the radiation imaging apparatus 1200 has the functions of the X-ray synchronization mode and the automatic trigger mode. The parameter flag_f of 1 indicates that the radiation imaging apparatus 1200 has the function of the X-ray synchronization mode. The parameter flag_f of 0 indicates that the radiation imaging apparatus 1200 cannot communicate with the imaging control apparatus 1300 or the radiation generation apparatus 1100.

The communication information about communication between the radiation imaging apparatus 1200 and the radiation generation apparatus 1100 and the function information about the imaging control apparatus 1300 are stored in the terminal side memory 1343 as an integer parameter flag_s. The parameter flag_s of 4 indicates that the radiation imaging apparatus 1200 and the radiation generation apparatus 1100 can communicate with each other. If the parameter flag_s is 3 or less, it indicates that the radiation imaging apparatus 1200 and the radiation generation apparatus 1100 cannot communicate with each other. The parameter flag_s of 3 indicates that the imaging control apparatus 1300 has the functions of performing the automatic trigger mode and the timer imaging. The parameter flag_s of 2 indicates that the imaging control apparatus 1300 can perform the timer imaging. The parameter flag_s of 1 indicates that the imaging control apparatus 1300 can perform the automatic trigger mode. The parameter flag_s of 0 indicates that the communication function between the FPD (radiation imaging apparatus 1200) side and the imaging control apparatus 1300 is disabled.

An integer parameter “mode” serving as an imaging mode (operation mode) setting value is determined based on the information about the parameter flag_f which indicates the function information about the radiation imaging apparatus side and the parameter flag_s which indicates the communication information about communication between the radiation imaging apparatus 1200 and the radiation generation apparatus 1100 and the function information about the imaging control apparatus 1300. If the parameter “mode” is 4, it indicates that the synchronous imaging mode (X-ray synchronization mode) is performed. The parameter “mode” of 3 indicates that the automatic trigger mode (X-ray automatic detection mode) and the timer imaging (X-ray manual synchronization mode) are performed. This corresponds to the X-ray asynchronous imaging mode illustrated in FIG. 7. The parameter “mode” of 2 indicates that the automatic trigger mode is performed. The parameter “mode” of 1 indicates that the timer imaging is performed. The parameter “mode” of 0 indicates that an image accumulation mode is performed. The parameter “mode” of null indicates that imaging using the radiation imaging apparatus 1200 of interest cannot be performed.

The image accumulation mode refers to a mode in which the radiation image data obtained by the radiation imaging apparatus 1200 according to imaging of radiations is sequentially stored into a removable memory 1280. To check the radiation image data, the memory 1280 is detached from the radiation imaging apparatus 1200. The memory 1280 is then connected to the imaging control apparatus 1300 to display and check images. The image accumulation mode is executed to perform imaging even if the communication with the radiation imaging apparatus 1200 cannot be established.

The exemplary embodiment may support only the four cases illustrated in FIG. 10 in the thick frames. In such cases, the radiation imaging apparatus 1200 may or may not have an automatic trigger function. Aside from the four cases in the thick frames, only the nine cases illustrated in the dotted frames may be supported. In such cases, a radiation imaging apparatus or apparatuses 1200 capable of only the X-ray synchronization mode and one(s) also capable of asynchronous imaging are both assumed to be used. The operation mode can thus be set because the information for making appropriate settings according to variations of the functions of the radiation imaging apparatus(es) 1200 in use is stored.

Such table information may be stored in the memory 1243 of the radiation imaging apparatus 1200, the terminal side memory 1343 of the imaging control apparatus 1300, and/or a notification unit memory 1643 of the notification unit 1600. The sensor control unit 1240 of the radiation imaging apparatus 1200, a control circuit of the imaging control apparatus 1300, and/or the notification control unit 1640 of the notification unit 1600 can refer to the table information, whereby the apparatus(es) or unit(s) can function as a management apparatus.

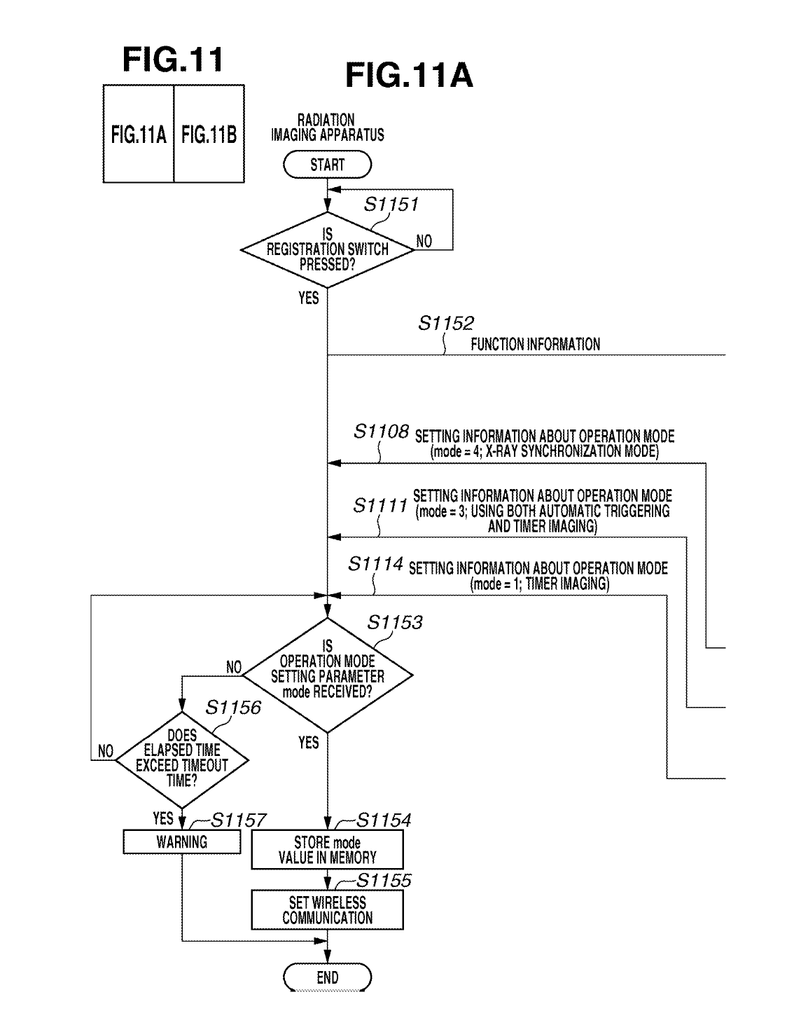

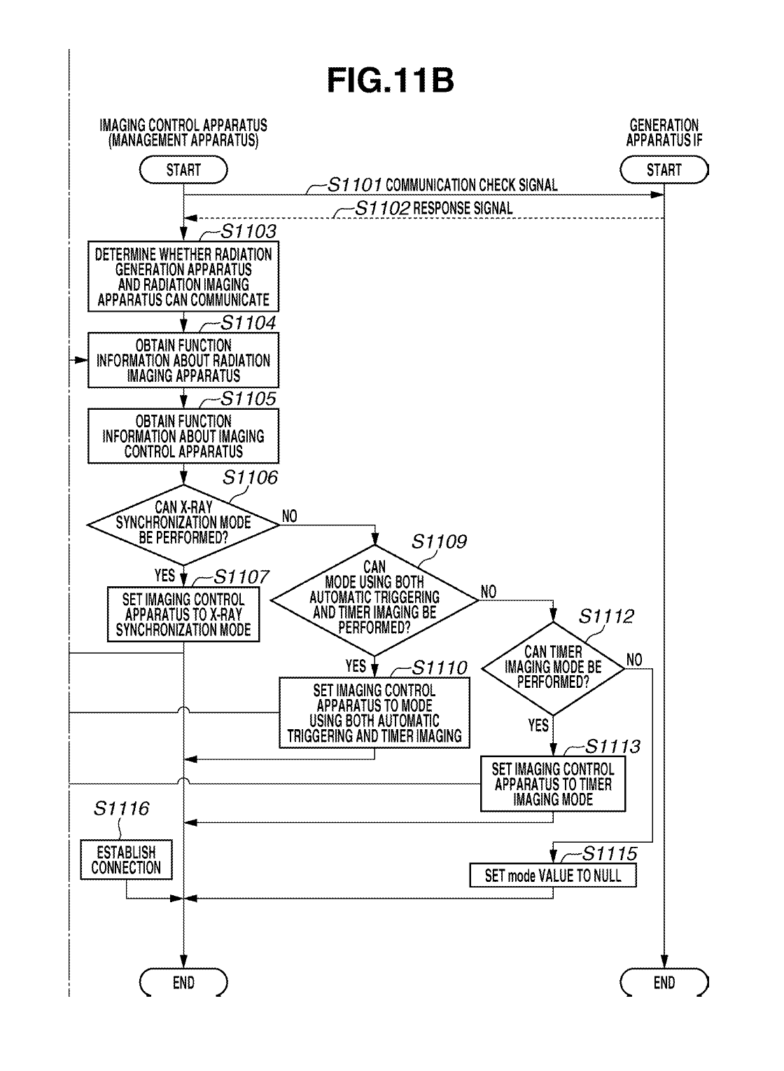

A flow of operation mode setting processing of the radiation imaging system according to the exemplary embodiment will be described with reference to FIG. 11.

In step S1101, the terminal side communication circuit 1395 of the imaging control apparatus (management apparatus) 1300 transmits a communication check signal such as a ping to the generation apparatus IF 1150. In step S1102, the terminal side communication circuit 1395 of the imaging control apparatus 1300 receives a response signal such as a pong from the generation apparatus IF 1150. If the generation apparatus IF 1150 is not connected with the wireless AP 1151 or if the generation apparatus IF 1150 is not activated, the terminal side communication circuit 1395 receives no response signal. If the connection between the generation control circuit 1130 and the generation apparatus IF 1150 has been established, the terminal side communication circuit 1395 receives a first response signal. If the generation apparatus IF 1150 is connected with the wireless AP 1151 but the connection between the generation control circuit 1130 and the generation apparatus IF 1150 has not been established, the generation apparatus IF 1150 transmits a second response signal different from the first response signal.

In step S1103, the control circuit of the imaging control apparatus 1300 determines whether the radiation generation apparatus 1100 and the radiation imaging apparatus 1200 can communicate with each other. The control circuit stores the determination result in the terminal side memory 1343 as communication information.

In step S1104, the control circuit of the imaging control apparatus 1300 obtains a communication connection request and the function information about the radiation imaging apparatus 1200 from the IR unit 1696 of the notification unit 1600 or the terminal side communication circuit 1395. The control circuit stores the obtained function information in the terminal side memory 1343.

In step S1105, the control circuit of the imaging control apparatus 1300 obtains the function information about the imaging control apparatus 1300. An example of the function information is information indicating whether the imaging control apparatus 1300 includes a software program corresponding to the automatic trigger mode and the X-ray manual synchronization mode.

In step S1106, the determination unit 1341 or the control circuit of the imaging control apparatus 1300 determines whether the X-ray synchronization mode can be performed. The determination in step S1106 and the determination in steps S1109 and S1112 are made based on the table information, which has been described above with reference to FIG. 10. If the determination unit 1341 or the control circuit determines that the X-ray synchronization mode can be performed (YES in step S1106), the processing proceeds to step S1107. If the determination unit 1341 or the control circuit determines that the X-ray synchronization mode cannot be performed (NO in step S1106), the processing proceeds to step S1109.

In step S1107, the terminal side setting unit 1360 sets the imaging control apparatus 1300 to the X-ray synchronization mode. The terminal side setting unit 1360 writes setting information to the terminal side memory 1343.

In step S1108, the IR unit 1696 or the terminal side communication circuit 1395 transmits a “mode” value of 4 serving as the setting information about the operation mode to the radiation imaging apparatus 1200 according to control of the control circuit.

In step S1109, the determination unit 1341 or the control circuit of the imaging control apparatus 1300 determines whether a mode using both automatic triggering and manual synchronization (asynchronous imaging mode) can be performed.

If the determination unit 1341 or the control circuit determines that the mode using both automatic triggering and manual synchronization can be performed (YES in S1109), in step S1110, the terminal side setting unit 1360 sets the imaging control apparatus 1300 to the mode using both automatic triggering and timer imaging. The terminal side setting unit 1360 writes a “mode” value of 3 serving as the setting information to the terminal side memory 1343.

In step S1111, the IR unit 1696 or the terminal side communication unit 1395 transmits the “mode” value of 3 serving as the setting information about the operation mode to the radiation imaging apparatus 1200 according to control of the control circuit.

If the determination unit 1341 or the control circuit determines that the mode using both automatic triggering and manual synchronization cannot be performed (NO in S1109), in step S1112, the determination unit 1341 or the control circuit of the imaging control apparatus 1300 determines whether the timer imaging mode can be performed.

If the determination unit 1341 or the control circuit determines that the timer imaging mode can be performed (YES in S1112), in step S1113, the terminal side setting unit 1360 sets the imaging control apparatus 1300 to the timer imaging mode. The terminal side setting unit 1360 writes a “mode” value of 1 serving as the setting information to the terminal side memory 1343.

In step S1114, the IR unit 1696 or the terminal side communication circuit 1395 transmits the “mode” value of 1 serving as the setting information about the operation mode to the radiation imaging apparatus 1200 according to control of the control circuit.

If the determination unit 1341 or the control circuit determines that the timer imaging mode cannot be performed (NO in S1112), in step S1115, the terminal side setting unit 1360 sets the “mode” value to null since none of the operation modes can be set. If the “mode” value has already been null, the terminal side setting unit 1360 performs control not to make a memory operation about the mode value. The terminal side communication circuit 1395 or the IR unit 1696 may transmit the “mode” value of null to the radiation imaging apparatus 1200 side in this step.

Step S1116 is performed after step S1107, S1110, or S1113. In step S1116, the terminal side setting unit 1360 establishes communication between the communication circuit 1295 and the wireless AP 1151.

An operation of the radiation imaging apparatus 1200 will be described below.

In step S1151, the sensor control unit 1240 of the radiation imaging apparatus 1200 determines whether a registration switch is pressed. The registration switch is an operation button for triggering communication of the infrared communication unit 1296 or the communication circuit 1295. If the sensor control unit 1240 determines that the registration switch is pressed (YES in step S1151), the processing proceeds to step S1152.

In step S1152, the infrared communication unit 1296 or the communication circuit 1295 transmits a wireless communication connection request and the function information about the radiation imaging apparatus 1200.

In step S1153, the sensor control unit 1240 repeats checking data received by the infrared communication unit 1296 or the communication circuit 1295, and continues determining whether the operation mode setting parameter “mode” is received. If the sensor control unit 1240 determines that the operation mode setting parameter “mode” is received (YES in step S1153), the processing proceeds to step S1154. If the sensor control unit 1240 determines that the operation mode setting parameter “mode” is not received (NO in step S1153), the processing proceeds to step S1156.

In step S1154, the setting unit 1260 stores the received “mode” value in the memory 1243, thereby setting the operation mode.

In step S1155, the sensor control unit 1240 makes wireless communication settings based on wireless communication parameters received through the infrared communication unit 1296 or the communication circuit 1295. The wireless communication parameters are parameters about communication with the wireless AP 1151.

If the communication circuit 1295 and the terminal side communication circuit 1395 are used to exchange the function information and the wireless communication parameters, such communication circuits 1295 and 1395 are implemented by performing communications based on the Wi-Fi Direct standard, for example.

In step S1156, the determination unit 1241 of the sensor control unit 1240 monitors the time elapsed from the pressing of the registration switch by using a function of the timer 1242. The determination unit 1241 determines whether the elapsed time exceeds a preset timeout time. If the determination unit 1241 determines that the elapsed time exceeds the timeout time (YES in step S1156), the processing proceeds to step S1157. If the determination unit 1241 determines that the elapsed time does not to exceed the timeout time (NO in step S1156), the processing returns to step S1153 to determine again whether the operation mode setting parameter “mode” is received.

In step S1157, the display control unit 1271 displays a warning on the display unit 1270. The contents of the warning indicate that the operation mode has failed to be set within a predetermined period. The display of the warning prompts the operator to check the system configuration and press the registration switch again. Alternatively, the communication circuit 1295 transmits an instruction signal for instructing the imaging control apparatus 1300 to notify the operator of the warning. According to control of the notification control unit 1640 or the terminal side display control unit 1271 which has received the instruction signal, the notification unit 1600 or the terminal side display unit 1370 notifies the user of the warning. The contents of the warning may include a sound notification aside from the foregoing.

The operation mode setting processing is completed by the processing described above.

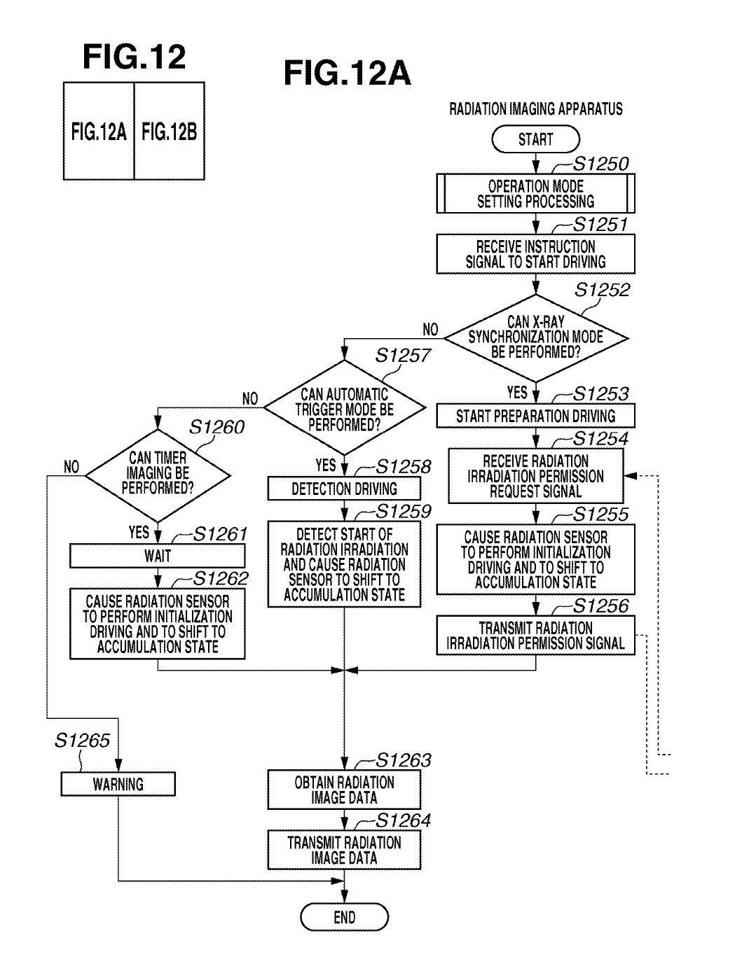

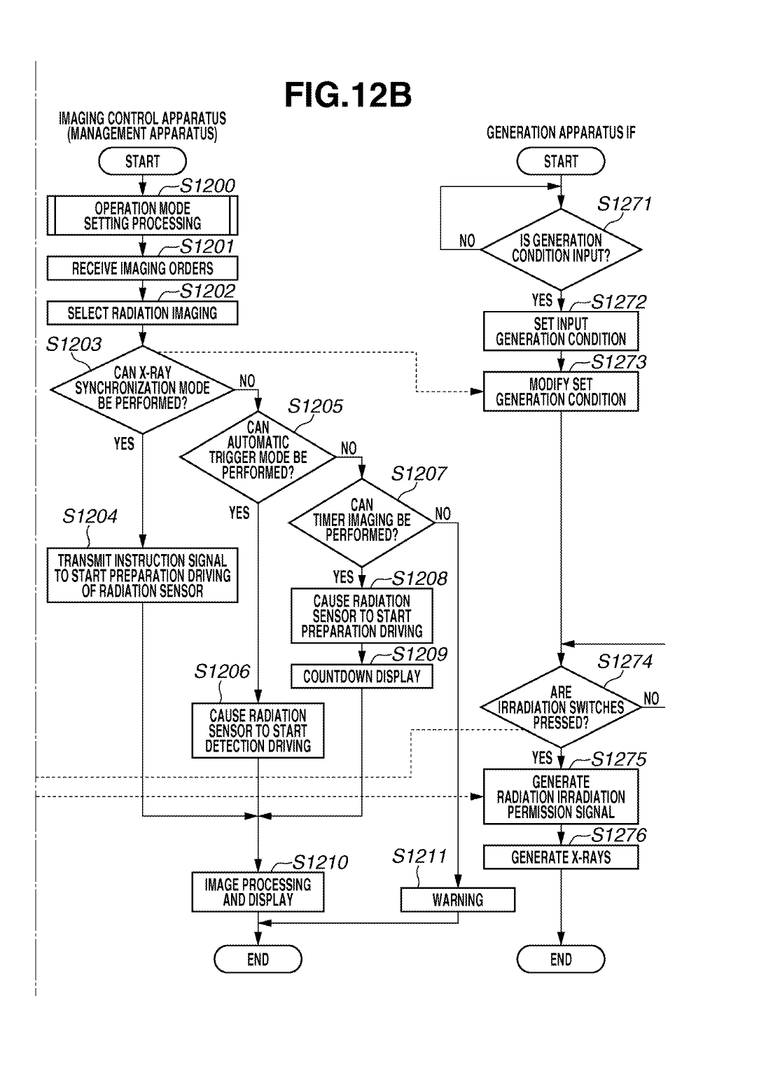

A flow of an imaging operation in a set operation mode will be described with reference to FIG. 12.

Steps S1200 and S1250 are the operation mode setting processing described above with reference to FIG. 11. A description thereof is thus omitted.

In step S1201, the terminal side communication circuit 1395 of the imaging control apparatus 1300 receives imaging orders from an RIS.

In step S1202, the imaging control apparatus 1300 selects an order included in the imaging orders according to an operation input via the terminal side operation unit 1350, and selects a single type of radiation imaging included in the order.

In step S1203, the terminal side determination unit 1341 refers to the “mode” value of the terminal side memory 1343 and determines whether the X-ray synchronization mode can be performed. If the terminal side determination unit 1341 determines that the X-ray synchronization mode can be performed (YES in step S1203), the processing proceeds to step S1204. In step S1204, the terminal side communication circuit 1395 transmits an instruction signal for starting preparation driving of the radiation sensor 1210. The preparation driving refers to initialization driving for repeating turning on/off the TFTs 1204 of the radiation sensor 1210 to output dark current data accumulated in the photoelectric conversion elements 1207. Such initialization driving is regularly performed at predetermined intervals until a radiation irradiation request is given.

If the terminal side determination unit 1341 determines that the X-ray synchronization mode cannot be performed (NO in step S1203), in step S1205, the terminal side determination unit 1341 determines whether the automatic trigger mode can be performed, according to the “mode” value of the terminal side memory 1343 and the type of the selected imaging. For example, the selected imaging may not be able to be performed in the automatic trigger mode because of an extremely low dose or an extremely short X-ray irradiation time. The terminal side determination unit 1341 thus refers to imaging information about the selected imaging and determines the compatibility with the automatic trigger mode based on predetermined dose information and information about irradiation time.

If the terminal side determination unit 1341 determines that the automatic trigger mode can be performed (YES in step S1205), the processing proceeds to step S1206. In step S1206, the terminal side communication circuit 1395 causes the radiation sensor 1210 to start detection driving for detecting the radiation irradiation. The detection driving refers to the driving described above with reference to FIG. 9. The foregoing initialization driving may be performed a predetermined number of times before the detection driving.

If the terminal side determination unit 1341 determines that the automatic trigger mode cannot be performed (NO in step S1205), in step S1207, the terminal side determination unit 1341 refers to the “mode” value of the terminal side memory 1343 and determines whether the timer imaging can be performed. If the terminal side determination unit 1341 determines that the timer imaging can be performed (YES in step S1207), the processing proceeds to step S1208. In step S1208, the terminal side communication circuit 1395 causes the radiation sensor 1210 to start preparation driving. The preparation driving here refers to repeating the foregoing initialization driving during a predetermined countdown time. After the preparation driving, the radiation sensor 1210 shifts to the accumulation state. In step S1209, the notification unit 1600 and the terminal side display unit 1370 display a countdown display for a predetermined period until accumulation is started.

In step S1210, the imaging control apparatus 1300 applies diagnostic image generation processing to radiation image data received from the radiation imaging apparatus 1200 via the terminal side communication circuit 1395. Examples of the diagnostic image generation processing include gradation conversion processing, dynamic range changing processing, and noise reduction processing. The terminal side display control unit 1371 displays the processed radiation image data on the terminal side display unit 1370.

If the terminal side determination unit 1341 determines that the timer imaging cannot be performed (NO in step S1207), in step S1211, the notification unit 1600 and the terminal side display unit 1370 of the imaging control apparatus 1300 display a warning indicating that none of the operation modes can be performed, according to control of the notification control unit 1640 and the terminal side display control unit 1371. The processing then ends. In such a manner, if none of the operation modes can be performed, a warning is displayed and the execution of the imaging operation is restricted to reduce radiation imaging with an inappropriate system configuration. Suppose that the operation mode setting processing illustrated in FIG. 11 fails to set any operation mode while the communication setting of the radiation imaging apparatus 1200 succeeds. Even in such a case, control is performed so that the imaging operation processing is disabled to reduce radiation imaging with an inappropriate system configuration.

A flow of processing of the radiation imaging apparatus 1200 will be described below.

In step S1251, the communication circuit 1295 receives a driving start instruction signal transmitted from the imaging control apparatus 1300 in step S1204, S1206, or S1208.

In step S1252, the determination unit 1241 refers to the “mode” value of the memory 1243 and determines whether the X-ray synchronization mode can be performed. If the determination unit 1241 determines that the X-ray synchronization mode can be performed (YES in step S1252), the processing proceeds to step S1253.

In step S1253, the drive circuit 1220 starts preparation driving according to an instruction from the sensor control unit 1240. The preparation driving here refers to the initialization driving for repeating turning on/off the TFTs 1204 of the radiation sensor 1210 to output dark current data accumulated in the photoelectric conversion elements 1207. Such initialization driving is regularly performed at predetermined intervals until a radiation irradiation request is given.

In step S1254, the radiation imaging apparatus 1200 receives a radiation irradiation permission request signal via the generation apparatus IF 1150, the wireless AP 1151, and the communication circuit 1295. In step S1255, the drive circuit 1220 causes the radiation sensor 1210 to perform the initialization driving a predetermined number of times, and then turns off the TFTs 1204 to causes the radiation sensor 1210 to shift to the accumulation state. In step S1256, the communication circuit 1295 transmits a radiation irradiation permission signal in response to the transition of the radiation sensor 1210 to the accumulation state caused by the drive circuit 1220. As a result, imaging is performed in the X-ray synchronization mode.

If the determination unit 1241 determines that the X-ray synchronization mode cannot be performed (NO in step S1252), in step S1257, the determination unit 1241 refers to the “mode” value of the memory 1243 and determines whether the automatic trigger mode can be performed. If the determination unit 1241 determines that the automatic trigger mode can be performed (YES in step S1257), the processing proceeds to step S1258. In step S1258, the drive circuit 1220 causes the radiation sensor 1210 to perform detection driving. The detection driving here refers to the driving described above with reference to FIG. 9. The foregoing initialization driving may be performed a predetermined number of times before the detection driving. In step S1259, the radiation detection circuit 1221 detects the start of radiation irradiation by the radiation generation apparatus 1100. Upon detecting the start of radiation irradiation, the radiation detection circuit 1221 inputs a detection signal to the drive circuit 1220, whereby the radiation sensor 1210 shifts to the accumulation state. As a result, imaging is performed in the automatic trigger mode (X-ray automatic detection mode).

If the determination unit 1241 determines that the automatic trigger mode cannot be performed (NO in step S1257), in step S1260, the determination unit 1241 refers to the “mode” value of the memory 1243 and determines whether the timer imaging can be performed. If the determination unit 1241 determines that the timer imaging can be performed (YES in step S1260), the processing proceeds to step S1261. In step S1261, the drive circuit 1220 waits for a predetermined wait time. The wait time here corresponds to the countdown time of the countdown display in step S1209. As described above, the radiation sensor 1210 may repeat the initialization driving during the wait.

In step S1262, the drive circuit 1220 causes the radiation sensor 1210 to perform the initialization driving a predetermined number of times, and then causes the radiation sensor 1210 to shift to the accumulation state. As a result, imaging is performed in the timer imaging mode (X-ray manual synchronization mode).

After the end of step S1256, S1259, or S1262, the processing proceeds to step S1263. In step S1263, the reading circuit 1230 reads an electrical signal corresponding to the charge accumulated in the radiation sensor 1210 in the accumulation state to obtain radiation image data. In step S1264, the communication circuit 1295 transmits the radiation image data to the imaging control apparatus 1300. The transmitted radiation image data is subjected to the image processing and display processing in the processing of step S1210.

If the determination unit 1241 determines that the timer imaging cannot be performed (NO in step S1260), in step S1265, the display control unit 1271 displays a warning on the display unit 1270. The contents of the warning indicate that the imaging using the radiation imaging apparatus 1200 cannot be performed. The display of the warning prompts the user to check the system configuration and press the registration switch again. Alternatively, the communication circuit 1295 transmits an instruction signal for instructing the imaging control apparatus 1300 to notify the user of the warning. According to control of the notification control unit 1640 or the terminal side display control unit 1271 which has received the instruction signal, the notification unit 1600 or the terminal side display unit 1370 notifies the user of the warning. The contents of the warning may include a sound notification aside from the foregoing.

A flow of processing of the radiation generation apparatus 1100 will be described below.

In step S1271, the generation control circuit 1130 repeats determination processing for determining whether a generation condition is input according to an operation input via the generation apparatus operation unit 1145. If the generation control circuit 1130 determines that a generation condition is input (YES in step S1271), the processing proceeds to step S1272. In step S1272, the generation control circuit 1130 sets the input generation condition. In step S1273, if the imaging control apparatus 1300 and the radiation generation apparatus 1100 can communicate with each other, the generation control circuit 1130 receives information about a predetermined generation condition corresponding to the imaging order received by the imaging control apparatus 1300 or a generation condition input and modified via the terminal side operation unit 1350 on the imaging control apparatus 1300 side. The generation control circuit 1130 receives such information via the generation apparatus IF 1150, and modifies the set generation condition.

In step S1274, the generation control circuit 1130 determines whether the first switch 1141 and the second switch 1142 of the irradiation switch 1140 are pressed. The generation control circuit 1130 repeats the determination processing until the first and second switches 1141 and 1142 are pressed. If generation control circuit 1130 determines that the first and second switches 1141 and 1142 are pressed (YES in step S1274), the generation control circuit 1130 transmits a radiation irradiation permission request signal via the generation apparatus IF 1150, and receives a permission signal. If the X-ray synchronization mode using the generation apparatus IF 1150 is not set, like when the generation apparatus IF 1150 is not connected, the permission signal is constantly input to the generation control circuit 1130. In such a case, the processing proceeds to step S1275 without waiting for the reception of the permission signal from outside.

In step S1275, the generation control circuit 1130 generates a radiation irradiation permission signal.

In step S1276, the generation control circuit 1130 controls the high-voltage power supply 1120 and the radiation source 1110 to generate X-rays.

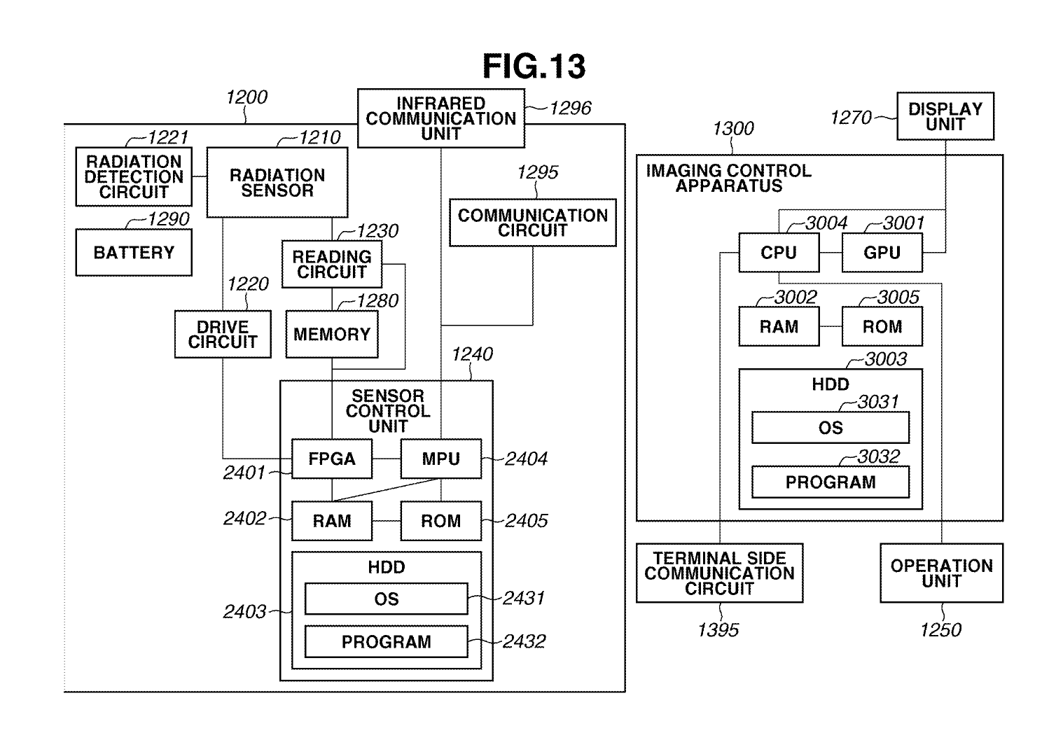

FIG. 13 illustrates a hardware configuration example of the radiation imaging apparatus 1200 and the imaging control apparatus 1300 according to the foregoing exemplary embodiment. Similar components or units to those of the foregoing example are designated by the same reference numerals. A description thereof may be omitted.

The sensor control unit 1240 includes an FPGA 2401, a RAM 2402, an HDD 2403, a micro processing unit (MPU) 2404, and a ROM 2405. The FPGA 2401 mainly controls the drive circuit 1220 and the reading circuit 1230. The MPU 2404 is a circuit for controlling the operation of the radiation imaging apparatus 1200 in an integrated manner. The MPU 2404 controls the components of the radiation imaging apparatus 1200 by executing instructions included in a program or programs stored in the ROM 2405 and/or the HDD 2403. The processing according to the foregoing exemplary embodiment is thus implemented. The RAM 2402 is a work memory of the MPU 2404. The HDD 2403 stores various types of setting data, as well as an operating system (OS) 2431 and a program 2432 which runs on the OS 2431. The program 2432 is a program for implementing the functions of the hardware illustrated in FIG. 13 and the processing illustrated in the flowcharts in FIGS. 11 and 12. The MPU 2404 executes the program 2432 to implement the functions and processing.

The imaging control apparatus 1300 includes a graphics processing unit (GPU) 3001, a RAM 3002, an HDD 3003, a CPU 3004, and a ROM 3005. The CPU 3004 is a circuit for controlling the hardware of the imaging control apparatus 1300 and units connected thereto in an integrated manner. The CPU 3004 controls the components of the imaging control apparatus 1300 by executing instructions included in programs stored in the ROM 3005 and/or the HDD 3003. The RAM 3002 is a work memory of the CPU 3004. The HDD 3003 stores various types of setting data, as well as an OS 3031 and a program 3032 which runs on the OS 3031. The program 3032 is a program for implementing the functions of the hardware illustrated in FIG. 13 and the processing illustrated in the flowcharts in FIGS. 11 and 12. The CPU 3004 executes the program 3032 to implement the functions and processing. The GPU 3001 is a dedicated circuit mainly for performing image processing. The GPU 3001 processes received image data according to instructions from the CPU 3004.

If the MPU 2404 or the CPU 3004 performs functions implemented by the FPGA 2401, a software program corresponding to the hardware description language used to implement the FPGA 2401 is provided and stored in the HDD 2403 or 3004 as the program 2432 or 3032. The MPU 2404 or the CPU 3004 executes, sequentially or in parallel, instructions included in the stored program 2432 or 3032, thereby implementing the foregoing processing illustrated in the flowcharts in FIGS. 11 and 12. If hardware performs the functions implemented by the MPU 2404 or the CPU 3004 and the program 2432 or 3032, a program described in the hardware description language corresponding to the program 2432 or 3032 is generated, from which FPGA configuration data is generated for implementation.

The significance of the foregoing exemplary embodiment will be described. Radiation imaging apparatuses may have different functions. For example, a radiation imaging apparatus having a radiation automatic detection function and one having no such function may be simultaneously used for radiation imaging. As for radiation generation apparatuses, some have a function for synchronization control on radiation irradiation timing and some do not. For example, a radiation imaging room equipped with a radiation imaging apparatus may have the function for synchronization control on radiation irradiation timing. A portable radiation generation apparatus (mobile X-ray apparatus) for CR may not have the function for synchronization control. Suppose that a radiation imaging apparatus having no radiation automatic detection function is used in combination with a radiation generation apparatus having no function for synchronization control on radiation irradiation timing. In such a case, the radiation imaging apparatus without the radiation automatic detection function fails to obtain a radiation image even if radiations are emitted in an interlocking manner with a radiation irradiation switch of the radiation generation apparatus. As a result, the patient undergoes needless exposure.

Suppose that a radiation imaging apparatus that can select the radiation automatic detection function and the radiation synchronization control function is used in combination with a radiation generation apparatus that can select the function for synchronization control on the radiation irradiation timing with the radiation imaging apparatus. In such a case, the operator needs to set the operation modes of the respective apparatuses according to the functions to use. The operation modes of the radiation imaging apparatus and the radiation generation apparatus both need to be properly set, or a radiation image cannot be obtained while the patient undergoes needless exposure. As the degree of freedom of combination between the radiation imaging apparatus and the radiation generation apparatus increases, there is the problem of needless exposure on the patient depending on the combination of the functions of the radiation imaging apparatus and the radiation generation apparatus. If the operation modes of the radiation imaging apparatus and the radiation generation system are selectable, complicated operations are needed to set the operation modes each time the operator changes the combination of the apparatuses. There is also another problem that re-imaging is needed if erroneous settings are made.

In view of such problems, the present exemplary embodiment can prevent re-imaging due to the combination of the functions of the radiation imaging apparatus and the radiation generation apparatus, and eliminate complicated settings to reduce setting errors.

The foregoing exemplary embodiments each have been described as a concrete example of implementation of an exemplary embodiment of the present invention. The technical scope of exemplary embodiments of the present invention should not be considered to be limited to the foregoing exemplary embodiments. Exemplary embodiments of the present invention may be practiced in various combinations without departing from the technical concept or main features thereof.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the foregoing exemplary embodiments, the radiation imaging system is controlled by using the communication information about the radiation generation apparatus and the function information about the radiation imaging apparatus, whereby an appropriate operation can be achieved according to the combination of the radiation imaging apparatus and the radiation generation apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-044402 filed Mar. 6, 2013, which is hereby incorporated by reference herein in its entirety.

What is claimed is:

1. A management apparatus for managing radiation imaging including a radiation imaging apparatus and a radiation generation apparatus, the management apparatus comprising:
an obtaining unit configured to obtain function information about the radiation imaging apparatus and communication information about communication between the radiation imaging apparatus and the radiation generation apparatus;
an operation setting unit configured to set an operation mode of the radiation imaging apparatus based on the function information or communication information obtained by the obtaining unit; and
a control unit configured to perform control corresponding to radiation imaging based on the set operation mode.

2. The management apparatus according to claim 1, wherein, if the radiation imaging apparatus is able to detect a start of radiation irradiation by using a detection circuit, a setting is made to cause the radiation imaging apparatus to operate in an automatic trigger mode in which a radiation sensor of the radiation imaging apparatus shifts to an accumulation state in response to detection of the radiation irradiation by the detection circuit.

3. The management apparatus according to claim 1, further comprising a determination unit configured to determine whether the radiation imaging apparatus and the radiation generation apparatus are able to communicate with each other,
wherein, if the determination unit determines that the radiation imaging apparatus and the radiation generation apparatus are not able to communicate with each other, the operation setting unit is configured to make a setting to cause the radiation imaging apparatus to operate in a timer imaging mode in which a radiation sensor of the radiation imaging apparatus shifts to an accumulation state in response to a lapse of a predetermined time.

4. The management apparatus according to claim 1, further comprising a determination unit configured to determine whether the radiation imaging apparatus and the radiation generation apparatus are able to communicate with each other,
wherein, if the determination unit determines that the radiation imaging apparatus and the radiation generation apparatus are able to communicate with each other, the operation setting unit is configured to make a setting to cause the radiation imaging apparatus to operate in a synchronization mode in which timing of radiation generation is controlled by synchronized communication between the radiation imaging apparatus and the radiation generation apparatus.

5. The management apparatus according to claim 3, wherein, if the radiation imaging apparatus is able to detect a start of radiation irradiation by using a detection circuit and the determination unit determines that the radiation imaging apparatus and the radiation generation apparatus are able to communicate with each other, the operation setting unit is configured to make a setting to cause the radiation imaging apparatus to operate in a synchronization mode in which timing of radiation generation is controlled by synchronized communication between the radiation imaging apparatus and the radiation generation apparatus.

6. The management apparatus according to claim 1, further comprising a notification unit configured to notify, if the radiation imaging apparatus is not able to detect a start of radiation irradiation and the radiation imaging apparatus and the radiation generation apparatus are not able to communicate with each other, that radiation imaging by the radiation imaging apparatus is not able to be performed.

7. The management apparatus according to claim 1, wherein, if the radiation imaging apparatus is not able to detect a start of radiation irradiation and the radiation imaging apparatus and the radiation generation apparatus are not able to communicate with each other, a setting is made to cause the radiation imaging apparatus to operate in an image accumulation mode in which radiation image data obtained by a plurality of times of radiation imaging by the radiation imaging apparatus is stored in a memory of the radiation imaging apparatus.

8. The management apparatus according to claim 1, wherein the control unit is configured to instruct transmission, to the radiation imaging apparatus, of a signal for causing the radiation imaging apparatus to start predetermined driving, in response to either one of an operator's instruction and an external input.

9. The management apparatus according to claim 1, wherein the control unit is configured to instruct the radiation imaging apparatus to start predetermined initialization driving in response to an external input and to transmit to the radiation generation apparatus a signal for permitting radiation generation in response to an end of the initialization driving.

10. The management apparatus according to claim 1, wherein the control unit is configured to cause the radiation imaging apparatus to start driving for detecting radiation irradiation in response to either one of an operator's instruction and an external input.

11. The management apparatus according to claim 1, wherein the control unit is configured to start time measuring in response to either one of an operator's instruction and an external input, and to cause a radiation sensor of the radiation imaging apparatus to shift to an accumulation state in response to a lapse of a predetermined time from the start of the time measuring.

12. The management apparatus according to claim 1, wherein the control unit is configured to output, in response to either one of an operator's instruction and an external input, a signal for causing a display unit to display that radiation irradiation is to be started.

13. The managing apparatus according to claim 1, further comprising a communication setting unit configured to perform communication setting between the radiation imaging apparatus of which the operation mode is set and the management apparatus,
wherein, if no operation mode is set by the operation setting unit, the communication setting unit is configured to not perform the communication setting.

14. The management apparatus according to claim 1, further comprising a memory configured to store table information in which a combination of the function information about the radiation imaging apparatus and the communication information about communication between the radiation imaging apparatus and the radiation generation apparatus is associated with the operation mode of the radiation imaging apparatus.

15. The management apparatus according to claim 1, wherein the management apparatus is configured to be mounted in a casing of the radiation imaging apparatus.

16. The management apparatus according to claim 1, wherein, if the radiation imaging apparatus is not able to detect a start of radiation irradiation and the radiation imaging apparatus and the radiation generation apparatus are not able to communicate with each other, the control unit is configured to prohibit the radiation imaging apparatus from performing radiation imaging.

17. The management apparatus according to claim 1, wherein the control unit is configured to cause the radiation imaging apparatus not to transmit a communication parameter for performing communication between the radiation imaging apparatus and an imaging control apparatus.

18. A management apparatus for managing radiation imaging by a radiation imaging apparatus, the management apparatus comprising:
a function obtaining unit configured to obtain function information about the radiation imaging apparatus;
a determination unit configured to determine, based on the function information, whether the radiation imaging apparatus includes a detection circuit configured to detect radiation irradiation; and
a control unit configured to, if the radiation imaging apparatus does not include the detection circuit and is determined to be unable to communicate with a radiation generation apparatus, perform at least either a control to prohibit the radiation imaging apparatus from performing the radiation imaging or a control to cause the radiation imaging apparatus not to transmit a communication parameter for performing communication between the radiation imaging apparatus and an imaging control apparatus.

19. A radiation imaging system including a radiation imaging apparatus and an imaging control apparatus, the radiation imaging system comprising:
a function obtaining unit configured to obtain function information about the radiation imaging apparatus;
a communication obtaining unit configured to obtain communication information about communication between the radiation imaging apparatus and a radiation generation apparatus;
an operation setting unit configured to set an operation mode of the radiation imaging apparatus based on the obtained function information or communication information; and
a control unit configured to perform control corresponding to radiation imaging based on the set operation mode.

20. The radiation imaging system according to claim 19,
wherein the radiation imaging apparatus includes an operation button, and
wherein the function obtaining unit is configured to obtain the function information about the radiation imaging apparatus in response to operation of the operation button.

21. The radiation imaging system according to claim 20,
wherein the radiation imaging apparatus and the imaging control apparatus each further include an infrared communication unit, and
wherein the radiation imaging apparatus and the imaging control apparatus are configured to perform infrared communication in response to the operation of the operation button, whereby information about an operation mode according to the function information and the communication information is transmitted from the imaging control apparatus to the radiation imaging apparatus.

22. A method for managing radiation imaging by a radiation imaging system including a radiation imaging apparatus and a radiation generation apparatus, the method comprising:
obtaining function information about the radiation imaging apparatus;
obtaining communication information about communication between the radiation imaging apparatus and the radiation generation apparatus;
setting an operation mode of the radiation imaging apparatus based on the obtained function information or communication information; and
performing control corresponding to radiation imaging based on the set operation mode.

23. A method for managing a radiation imaging apparatus, the method comprising:
obtaining function information about the radiation imaging apparatus;
determining, based on the function information, whether the radiation imaging apparatus includes a detection circuit configured to detect radiation irradiation; and
performing at least either a control to prohibit the radiation imaging apparatus from performing radiation imaging or a control to cause the radiation imaging apparatus not to transmit a communication parameter for performing communication between the radiation imaging apparatus and an imaging control apparatus if the radiation imaging apparatus does not include the detection circuit and is determined to be unable to communicate with a radiation generation apparatus.

24. A computer-readable storage medium storing a program for causing a computer to perform the method according to claim 22.

25. A computer-readable storage medium storing a program for causing a computer to perform the method according to claim 23.
